SEQUENCE LISTING (4 sequences)

SEQ ID NO: 1 - DNA, 660 bp, Artificial sequence
  Synthetic Sequence
atggagaaaaaaatcactggatataccaccgttgatatatcccaatgacatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgagatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaa

SEQ ID NO: 2 - DNA, 660 bp, Artificial sequence
  Synthetic sequence
atggagaaaaaaatcactggatataccaccgttgatatatcccaacgacatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaa

SEQ ID NO: 3 - DNA, 660 bp, Artificial sequence; identical to SEQ ID NO: 2
  Synthetic sequence

SEQ ID NO: 4 - DNA, 660 bp, Artificial sequence; identical to SEQ ID NO: 2
  Synthetic sequence

(19) United States
(12) Patent Application Publication
(10) Pub. No.: US 2024/0052357 A1
(43) Pub. Date: Feb. 15, 2024

(54) AUTONOMOUS INDUCIBLE DIRECTED EVOLUTION OF COMPLEX PATHWAYS

(71) Applicant: North Carolina State University, Raleigh, NC (US)

(72) Inventors: Nathan Crook, Raleigh, NC (US); Ibrahim Al'Abri, Raleigh, NC (US)

(21) Appl. No.: 17/766,307

(22) PCT Filed: Oct. 2, 2020
(86) PCT No.: PCT/US2020/054100

§ 371 Date: Apr. 4, 2022

Related U.S. Application Data
(60) Provisional application No. 62/910,035, filed on Oct. 3, 2019.

(51) Int. Cl. C12N 15/70; C12N 15/01; C12N 1/06

(52) U.S. Cl. CPC C12N 15/70; C12N 15/01; C12N 1/06

(57) ABSTRACT

A method for directed evolution in a microbe, the method comprising: introducing into a first host cell a propagation deficient phage genome and a vector comprising a target gene sequence to be mutated and a phage propagation component responsive to induce lysis of the first host cell and to provide for propagation of the phage genome; exposing the first host cell to a mutagenesis agent; inducing lysis of the first host cell and phage propagation to produce a lysate comprising phage particles comprising the target gene sequence; and infecting a second host cell with the lysate. Systems and kits for practicing the method are also disclosed.

### CROSS-REFERENCE TO RELATED APPLICATIONS

This application claims benefit of U.S. Provisional Patent Application Ser. No. 62/910,035, filed Oct. 3, 2019, which is herein incorporated by reference in its entirety.

### TECHNICAL FIELD

The presently disclosed subject matter relates to methods and systems for directed evolution of complex pathways.

### BACKGROUND

Many important phenotypes emerge from the interactions between multiple genes (Harvey et al., Nature Reviews Drug Discovery, 14:111-129 (2015); Crook, N. & Alper, H. S., Engineering Complex Phenotypes in Industrial Strains 1-33 (2012)). These “complex” phenotypes have traditionally encompassed small molecule biosynthesis (Lee, S. Y. et al., Nature Catalysis, 2:18-33 (2019)), tolerance to inhibitors (Pham et al., Nature Communications, 8:411 (2017)), and growth in new habitats (Ravikumar et al., Cell 175:1946-1957 (2018)). However, advances in synthetic biology and metabolic engineering have revealed that even supposedly “simple” phenotypes, such as production of a recombinant protein, become complex as higher performance is desired. This is because auxiliary cellular functions, such as chaperone proteins, cell wall synthesis, and secretion machinery can become limiting in these contexts. Clearly, engineering these “systems-level” phenotypes requires systems-level techniques.

In bacteria, complex phenotypes can be accessed via adaptive evolution (Crook et al. 2019, Cell Host Microbe 25:499-512; Gleizer et al. 2019, Cell 179:1255-1263; Wong et al. 2018, Nat. Biotechnol. 36:614-623 (2018)) or iterative genome-wide expression perturbation screens (e.g. asRNA (Meng, Jia, et al 2012 FEMS microbiology letters 329.1 (2012): 45-53; Georg, J., & Hess, W. R. 2011, Microbiology and Molecular Biology Reviews, 75(2), 286-300; and CRISPRi/a (Bikard et al. 2012 Cell host & microbe, 12(2), 177-186; Reis et al. 2019, Nat. Biotechnol. 37, 1294-1301; Dong et al. 2018, Nat. Commun. 9, 2489)). One downside of adaptive evolution is the accumulation of genomic hitchhiker mutations, a feature that is particularly troublesome for biosensor-coupled screens and makes learning from these experiments very time-consuming. For asRNA and CRISPRi/a, the researcher is limited to sampling changes to expression space, rather than the much larger space of protein bioactivity.

For these reasons, directed evolution is a promising alternative, since it directs mutations to defined DNA sequences and samples the entire sequence space in that region. However, due to the limited length of DNA that can be evolved using most methods, it has been difficult to apply directed evolution to complex phenotypes. For example, traditional error-prone PCR-based libraries are effectively limited to sequences <10 kb in length due to reductions in polymerase processivity, cloning efficiency, and transformation rate above this size. Although recent methods for directed evolution in bacteria have overcome the traditionally laborious and costly steps for generating error-prone libraries (e.g. phage-assisted continuous and non-continuous evolution (PACE, PANCE)(Roth et al. ACS Synth. Biol. 8:796-806 (2019); Suzuki, T. et al., Nat. Chem. Biol. 13:1261-1266 (2017); Esvelt et al., Nature 472:499-503 (2011); and Phage-and-Robotics-Assisted Near-Continuous Evolution (PRANCE)(DeBenedictis et al., bioRxiv 2020.04.01.021022 (2020)), they are limited to small regions of DNA (<5 kb) and couple the mutagenesis and screening steps. This is because the phage used in these techniques (M13) has a strict packaging limit (no more than 5 kb) and is engineered to replicate as soon as a certain threshold of biological activity has been reached. Indeed, although PACE has recently been used to evolve multigene pathways, the library size is significantly reduced (˜10\hat{ }5)(Johnston et al., Nat. Commun. 11:4202 (2020)). Thus, alternative approaches to directed evolution represent an ongoing need in the art.

### SUMMARY

In some embodiments, the presently disclosed subject matter provides a method for directed evolution in a microbe. In some embodiments, the method comprises: introducing into a first host cell a propagation deficient phage genome and a vector comprising a target gene sequence to be mutated and a phage propagation component responsive to induce lysis of the first host cell and to provide for propagation of the phage genome; exposing the first host cell to a mutagenesis agent; inducing lysis of the first host cell and phage propagation to produce a lysate comprising phage particles comprising the target gene sequence; and infecting a second host cell with the lysate. In some embodiments, the first host cell and the second host cell are each a bacterial cell.

In some embodiments, the phage genome comprises a temperate phage genome. In some embodiments, the temperate phage genome is a P1 phage genome. In some embodiments, the first host cell further comprises a second vector comprising an inducible mutagenesis sequence. In some embodiments, the mutagenesis agent is selected from the group consisting nucleotide analogues, nucleoside precursors, alkylating agents, cross-linking agents, genotoxins, and radiation. In some embodiments, the mutagenesis agent is a chemical mutagen.

In some embodiments, the method comprises screening for a selected function of a mutated target sequence. In some embodiments, the step of screening comprises at least one of a bacteriophage display system, an antibiotic resistance and an expression of a reporter gene.

In some embodiments, the method further comprises expressing an evolved protein or nucleic acid encoded by a mutated target sequence. In some embodiments, the method further comprises isolating a mutated target sequence. In some embodiments, the method comprises repeating said steps.

In some embodiments, a system or kit configured for carrying out a method in accordance with the presently disclosed subject matter is provided.

In some embodiments, provided is a kit for directed evolution in a microbe, the kit comprising: a propagation deficient phage genome and a phage propagation component responsive to induce lysis of a first host cell and to provide for propagation of the phage genome. In some embodiments, the kit comprises one or more of the following: a vector for a target gene sequence to be mutated, a first host cell for the propagation deficient phage genome and the phage propagation component, a second host cell for a lysate comprising phage particles, and a mutagenesis agent.

In some embodiments, the first host cell and the second host cell are each a bacterial cell. In some embodiments, the phage genome comprises a temperate phage genome. In some embodiments, the temperate phage genome is a P1 phage genome.

In some embodiments, the kit comprises a second vector comprising an inducible mutagenesis sequence. In some embodiments, the mutagenesis agent is selected from the group consisting nucleotide analogues, nucleoside precursors, alkylating agents, cross-linking agents, and genotoxins. In some embodiments, the kit comprises instructional material for a directed evolution method.

Accordingly, it is an object of the presently disclosed subject matter to provide Autonomous Inducible Directed Evolution (AIDE) methods, systems, and kits. This and other objects are achieved in whole or in part by the presently disclosed subject matter. An object of the presently disclosed subject matter having been stated above, other objects and advantages of the presently disclosed subject matter will become apparent to those of ordinary skill in the art after a study of the following description of the presently disclosed subject matter and non-limiting Examples and Figures.

### BRIEF DESCRIPTION OF THE DRAWINGS

The Figures provided herein have been included to provide guidance to one of ordinary skill in the art for practicing representative embodiments of the presently disclosed subject matter. In light of the present disclosure and the general level of skill in the art, those of skill can appreciate that the following Figures are intended to be exemplary only and that numerous changes, modifications, and alterations can be employed without departing from the scope of the presently disclosed subject matter.

FIG. 1 is a schematic of a representative Autonomous Inducible Directed Evolution (AIDE) approach in accordance with the presently disclosed subject matter.

FIGS. 2A through 2C are bar graphs showing optimization of phagemid infection and packaging levels. The phagemid infection/packaging level was increased by ˜49-fold compared to methods reported in prior studies. FIG. 2A shows how the physiological state of cells (E. coli C600) affects phagemid infection rate. FIG. 2B shows salt composition in growth medium influences phage absorption rate and cells stability. FIG. 2C is a bar graph showing P1kc::10 kb and P1kcΔCoi packaging/infection rates relative to wild-type P1.

FIG. 3 shows single and double stop codon reversion in CmR, including sequences of 3 colonies showing that both stop codons were reverted via AIDE. Double peaks show that not all plasmids in the cells were converted to functional codons. 3 replicates were evolved to gain chloramphenicol resistance, starting from a CmR gene containing either a single or double stop codon. FIG. 3 shows Sanger Sequences showing the reversion of two premature stop codons inserted in CmR at codons 16 and 85. Both codons were originally Trp (TGG). Double peaks indicate that the cells have two copies of the phagemids (one with a stop codon and one with a reverted stop codon). Sequences are presented as SEQ ID NOs.: 1-4 and in Table 4 herein below.

FIG. 4 is a plot showing Directed Evolution of sfGFP using AIDE. A phagemid containing sfGFP was evolved using AIDE to test the ability to perform multi-round evolution. The sfGFP phagemid went through 4 AIDE cycles, and the fluorescence of the cell population at each round was compared to the un-evolved phagemid, as well as negative and positive controls.

FIGS. 5A to 5D show AIDE overview and optimization. FIG. 5A shows that the effect of insert size in phagemid is negligible. 9 kbp, 12 kbp, 24 kbp, and 42 kbp phagemids were packaged in the same strain containing P1kc::10 kb and the same amount of phage lysate was used to infect wild type C600 E. coli. FIG. 5B shows the rate of reversion of single stop codons in CmR using AIDE. FIG. 5C is a schematic showing how different E. coli strains can be used for diversification and screening steps. FIG. 5D shows the efficiency of infection by phage lysate produced from 3 different E. coli strains (C600, MG1655 or Nissle) during infection of the same 3 strains. This heat map shows how different strains are better at producing phage and others are better at being infected.

FIGS. 6A through 6D show directed evolution of complex phenotypes. FIG. 6A is a schematic showing evolution of a phagemid containing sfGFP-pSC101 using AIDE. FIG. 6B is a plot showing the effect of verified mutations in pSC101 origin on the level of GFP expression, compared to an unevolved positive control. FIG. 6C is a schematic showing evolution of a tagatose pathway using AIDE. FIG. 6D is a plot of growth curves for isolated variants with verified mutations in the tagatose pathway after 2 AIDE cycles.

FIG. 7 is a bar graph showing how the multiplicity of infection (MOI) of P1::10 kb and P1ΔCoi and phagemid affect library size. Each dot represents one biological replicate.

FIG. 8 is a bar graph showing how the amounts of infected cells affect the library passaged in an AIDE cycle. 3 biological replicates of E. coli C600 cells were grown to OD 1 and concentrated to 1×, 2×, 3×, 5× and 10× and then infected with 3 phage lysates produced from E. coli C600.

FIG. 9 is a bar graph showing the effect of phagemid copy number on the phagemid packaging and infection rates as compared to P1kc::10 kb. Each dot represents one biological replicate.

### DETAILED DESCRIPTION

Directed evolution enables biological function to be engineered in the absence of detailed biochemical models, and often suggests novel or counterintuitive routes to improved function. Recent advances in methods for directed evolution have addressed the traditionally laborious and costly steps for generating error-prone libraries, which include molecular cloning and transformation. However, all methods for directed evolution developed to date are limited either to small regions of DNA (<10 kb), couple mutagenesis and screening steps, or allow the accumulation of off-target genomic mutations. The presently disclosed subject matter provides an approach referred to as Autonomous Inducible Directed Evolution (AIDE) to address these limitations. In representative, non-limiting embodiments, AIDE may harness P1 bacteriophage's ability to package plasmids up to 90 kb in size and transfer them efficiently to new Escherichia coli cells. Cells may also contain inducible mutation machinery (e.g. a re-engineered Mutagenesis Plasmid MP6⁵ (Badran, Ahmed H., and David R. Liu. Nature communications 6.1 (2015): 1-10.) (referred to herein as aTc-MP or ISA265 (pTet, tetR, DNAQ926, dam, seqA, emrR, ugi, cda1, CloDF13, KanR)) provided for facile mutagenesis of the entire 90 kb plasmid, of which ˜85 kb can comprise user-defined DNA. Mutated plasmids can then be transferred to new cells via P1 packaging, thus eliminating off-target genomic mutations before assays for improved phenotypes.

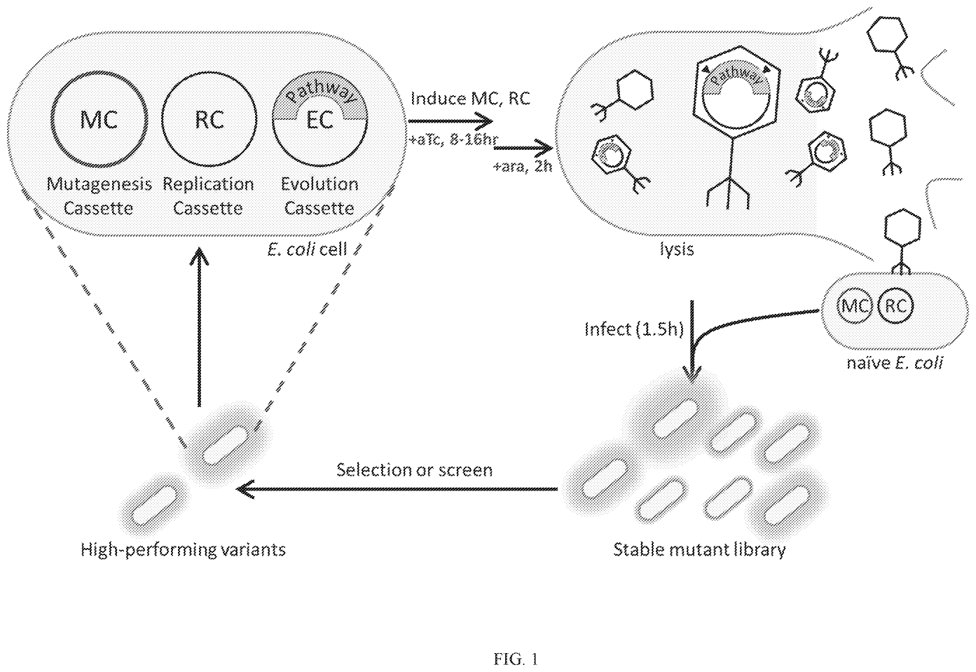

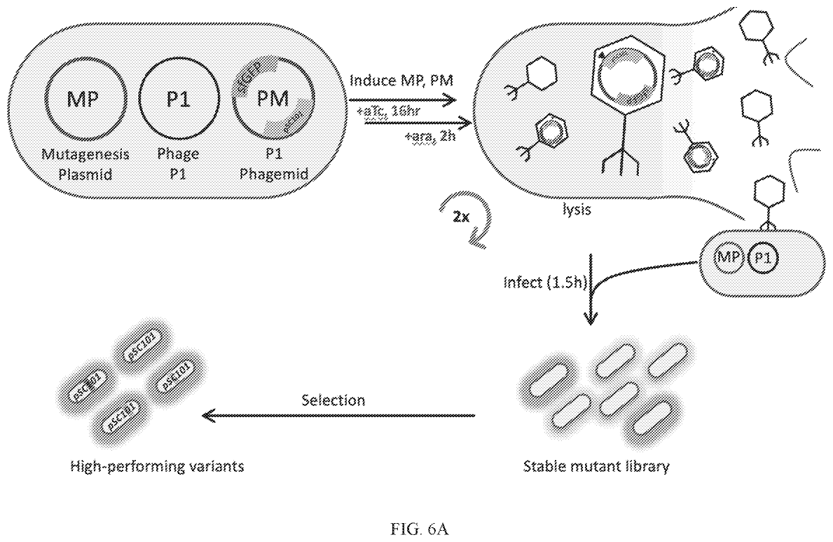

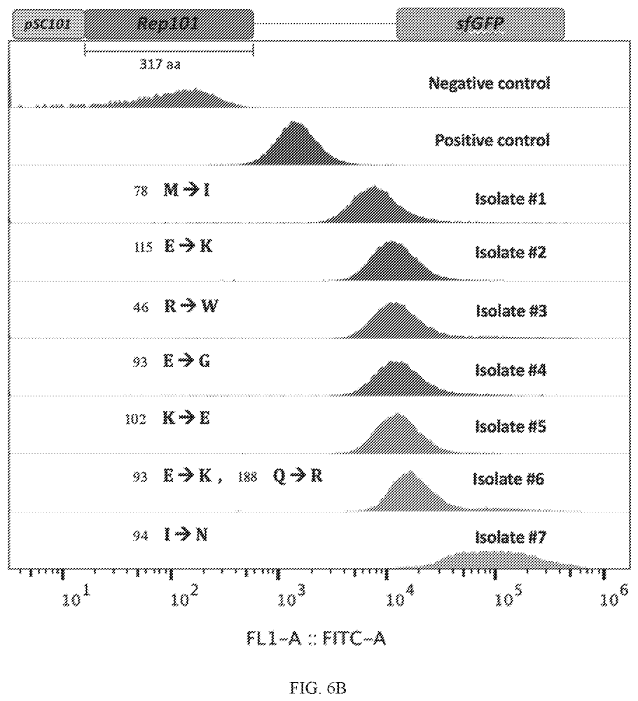

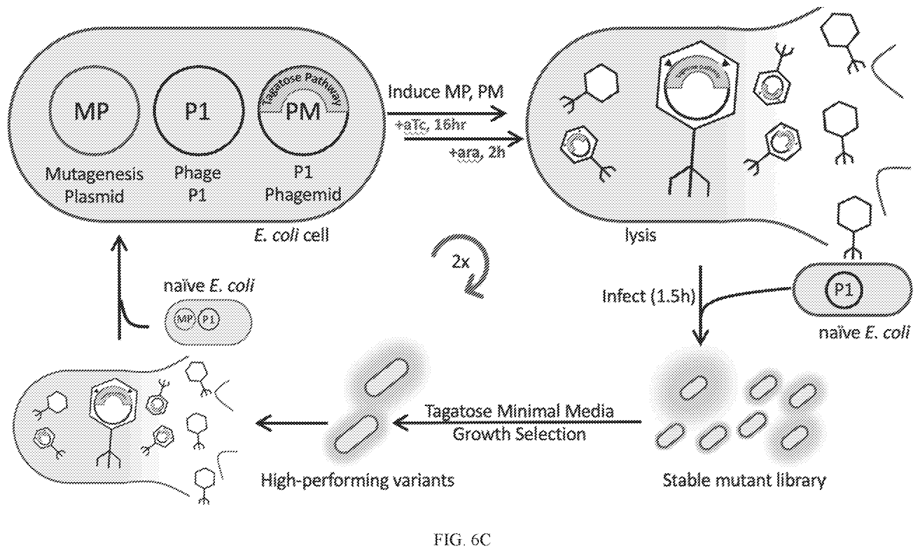

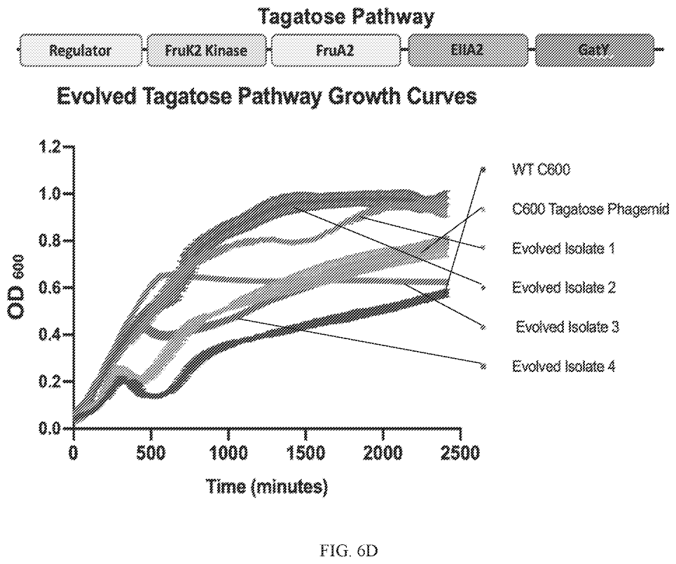

In representative, non-limiting embodiments, AIDE uses a temperate bacteriophage to package large plasmids and transfer them to naive cells after mutagenesis. Referring to FIGS. 1, 6A, and 6C, the AIDE workflow is both simple and flexible. Pathways of interest are assembled in a phagemid and transformed to a bacterium containing a helper phage. The master regulator for this phage is placed under inducible control. Next, mutagenesis is induced to create random mutations. Then, the phage lytic cycle is induced to initiate phagemid packaging and cell lysis. The resulting phage particles can then be applied to an unmutated strain, causing each recipient cell to express a different mutated copy of the DNA and allowing a subsequent screening or mutagenesis step.

Aspects of AIDE include autonomous generation of mutations to a specified DNA sequence by the bacterium itself, without the need for cloning and transformation each round; and periodic and automatable transfer of evolved material to fresh strains to eliminate accumulation of off-target mutations. Several benefits of this approach include but are not limited to (1) library size scales with the number of cultured bacteria, indicating that 10¹⁰ (10 mL of E. coli culture) or more mutated sequences can be easily generated; (2) the length of the sequence under selection (the “cargo”) can be up to 100 kb (for example, P1 packaging limit); (3) mutant libraries are generated easily, cheaply, and automatedly by adding chemical inducers to a bacterial culture and mixing the resulting lysate with fresh cells; (4) evolved DNA is decoupled from genomic adaptation through periodic lysis and re-infection; and (5) it is applicable to any screenable phenotype.

The presently disclosed subject matter will now be described more fully hereinafter with reference to the accompanying Examples and Figures, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art. Certain components in the figures are not necessarily to scale, emphasis instead being placed upon illustrating the principles of the presently disclosed subject matter (in some cases schematically).

### I. DEFINITIONS

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

While the following terms are believed to be well understood by one of ordinary skill in the art, the following definitions are set forth to facilitate explanation of the presently claimed subject matter.

The term “evolved” refers to a process of change that results in the production of new nucleic acids and polypeptides that retain at least some of the structural features or elements and/or functional activity of the parent nucleic acids or polypeptides from which they have developed. In some instances, the evolved nucleic acids or polypeptides have increased or enhanced activity compared with the parent. In some instances, the evolved nucleic acids or polypeptides have decreased or reduced activity compared with the parent.

The terms “nucleic acids,” “nucleic acid strand,” and “polynucleotide” refer to a polymeric form of nucleotides of any length, either ribonucleotides or deoxyribonucleotides. These terms include a single-, double- or triple-stranded DNA, genomic DNA, cDNA, genomic RNA, mRNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P—NH₂) or a mixed phosphoramidate-phosphodiester oligomer (Peyrottes et al. (1996) Nucleic Acids Res. 24: 1841-8; Chaturvedi et al. (1996) Nucleic Acids Res. 24: 2318-23; Schultz et al. (1996) Nucleic Acids Res. 24: 2966-73). A phosphorothioate linkage can be used in place of a phosphodiester linkage (Braun et al. (1988) J. Immunol. 141: 2084-9; Latimer et al. (1995) Molec. Immunol. 32: 1057-1064). In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The following are non-limiting examples of nucleic acid strands: a gene or gene fragment, exons, introns, genomic RNA, mRNA, tRNA, rRNA, ribozymes, cDNA, recombinant polynucleotides, branched polynucleotides, plasmids, vectors, isolated DNA of any sequence, and isolated RNA of any sequence. A nucleic acid strand may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A nucleic acid strand may be further modified after polymerization, such as by conjugation with a labeling component. Other types of modifications included in this definition are caps, and substitution of one or more of the naturally occurring nucleotides with an analog.

A “mutagenized nucleic acid” is a nucleic acid which has been physically altered as compared to a parental nucleic acid (e.g., such as a naturally occurring nucleic acid), e.g., by modifying, deleting, rearranging, or replacing one or more nucleotide residue in the mutagenized nucleic acid as compared to the parental nucleic acid.

A “transcribed” nucleic acid is a nucleic acid produced by copying a parental nucleic acid, where the parental nucleic acid is a different nucleic acid type than the copied nucleic acid. For example, an RNA copy of a DNA molecule (e.g., as occurs during classical transcription) or a DNA copy of an RNA molecule (e.g., as occurs during classical reverse transcription) can be a “transcribed nucleic acid” as that term is intended herein. Similarly, artificial nucleic acids, including peptide nucleic acids, can be used as either the parental or the copied nucleic acid (and artificial nucleotides can be incorporated into either parental or copied molecules). Copying can be performed, e.g., using appropriate polymerases, or using in vitro artificial chemical synthetic methods, or a combination of synthetic and enzymatic methods.

An “in vitro translation reagent” is a reagent which is necessary or sufficient for in vitro translation, or a reagent which modulates the rate or extent of an in vitro translation reaction, or which alters the parameters under which the reaction is operative. Examples include ribosomes, and reagents which include ribosomes, such as reticulocyte lysates, bacterial cell lysates, cellular fractions thereof, amino acids, t-RNAs, etc.

The terms “propagation component” and “propagation signal” are used interchangeably and refer to one or more proteins or nucleic acids that are required for phage replication, packaging or infection. The propagation component can comprise a phage packaging signal or a phage propagation signal.

The phrase “signal is functionally disabled” refers to a signaling pathway which has been altered so that a specific function is inactive. For example, the phage propagation signal can be disabled through the inactivation of one or more genes in the pathway, or inhibiting the binding of an essential element. “Phage packaging signal” refers to a stretch of residues recognized by the phage packaging proteins. “Phage propagation signal” is intended to include genes and functional RNAs involved in phage propagation.

A “translation product” is a product (typically a polypeptide) produced as a result of the translation of a nucleic acid. A “transcription product” is a product (e.g., an RNA, optionally including mRNA, or, e.g., a catalytic or biologically active RNA) produced as a result of transcription of a nucleic acid.

The term “random” refers to condition wherein events are determined by a probability distribution. The distribution may include a bias, e.g., dependent on the relative concentrations of starting material. For example, in one embodiment, the parental nucleic acid strands may include a biased amount of one species relative to another. The ligation of a mixture of fragments generated from such a pool of starting material can nevertheless be random.

The term “oligonucleotide,” as used herein refers to a nucleic acid polymer of about 5 to 140 nucleotides in length.

The term “protein,” as used herein refers to a sequence of amino acids that have a function and/or activity. Examples of activities of proteins include, but are not limited to, enzymatic activity, kinase activity, and binding activity, which can be shown through a variety of spectroscopic, radioactive, or direct binding assays which are known in the art. For example, see Sigma Aldrich for a collection of test kits and assays for biological activity.

The term “binds,” and “binding” refer to a physical interaction for which the apparent dissociation constant of two molecules is at least 0.1 mM. Binding affinities can be less than about 10 μM, 1 μM, 100 nM, 10 nM, 1 nM, 100 pM, 10 pM, and so forth. The term “ligand” refers to a compound which can be specifically and stably bound by a molecule of interest.

As used herein, “vector (or plasmid or phagemid)” refers to discrete elements that are used to introduce heterologous DNA into cells for either expression or replication thereof. Selection and use of such vehicles are well known within the skill of the artisan. An expression vector includes vectors capable of expressing DNA's that are operatively linked with regulatory sequences, such as promoter regions, that are capable of effecting expression of such DNA fragments. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, “a promoter region or promoter element” refers to a segment of DNA or RNA that controls transcription of the DNA or RNA to which it is operatively linked. The promoter region includes specific sequences that are sufficient for RNA polymerase recognition, binding and transcription initiation. This portion of the promoter region is referred to as the promoter. In addition, the promoter region includes sequences that modulate this recognition, binding and transcription initiation activity of RNA polymerase. These sequences may be cis acting or may be responsive to trans acting factors. Promoters, depending upon the nature of the regulation, may be constitutive or regulated.

As used herein, “operatively linked or operationally associated” refers to the functional relationship of DNA with regulatory and effector sequences of nucleotides, such as promoters, enhancers, transcriptional and translational stop sites, and other signal sequences. For example, operative linkage of DNA to a promoter refers to the physical and functional relationship between the DNA and the promoter such that the transcription of such DNA is initiated from the promoter by an RNA polymerase that specifically recognizes, binds to and transcribes the DNA. In order to optimize expression and/or in vitro transcription, it may be necessary to remove, add or alter 5′ untranslated portions of the clones to eliminate extra, potential inappropriate alternative translation initiation (i.e., start) codons or other sequences that may interfere with or reduce expression, either at the level of transcription or translation. Alternatively, consensus ribosome binding sites (for example, a Shine-Delgarno sequence for E. coli, as described by Shine and Delgarno, Nature. 1975 Mar. 6; 254(5495):34-8) can be inserted immediately 5′ of the start codon and may enhance expression. The desirability of (or need for) such modification may be empirically determined.

The term “mutagenesis agent” can refer to a vector (or plasmid) comprising an inducible mutagenesis sequence. The term “mutagenesis agent” can also refer to a chemical mutagen or to radiation using, for example, UV, gamma-irradiation, X-rays, and fast neutrons. Chemical mutagens are classifiable by chemical properties, e.g., alkylating agents, cross-linking agents, genotoxins, etc. The following chemical mutagens are useful, as are others not listed here, according to the presently disclosed subject matter. N-ethyl-N-nitrosourea (ENU), N-methyl-N-nitrosourea (MNU), procarbazine hydrochloride, chlorambucil, cyclophosphamide, methyl methanesulfonate (MMS), ethyl methanesulfonate (EMS), diethyl sulfate, acrylamide monomer, triethylene melamin (TEM), melphalan, nitrogen mustard, vincristine, dimethylnitrosamine, N-methyl-N′-nitro-Nitrosoguani-dine (MNNG), 7,12 dimethylbenz (a) anthracene (DMBA), ethylene oxide, hexamethylphosphoramide, bisulfan. Chemical mutagens useful in the present invention can also include, for example, sodium bisulfite, nitrous acid, hydroxylamine, hydrazine or formic acid. Other agents which are analogues of nucleotide or nucleoside precursors include nitrosoguanidine, 5-bromouracil, 2-aminopurine, 5-formyl uridine, isoguanosine, acridine and of N4-aminocytidine, N1-methyl-N4-aminocytidine, 3,N4-ethenocytidine, 3-methylcytidine, 5-hydroxycytidine, N4-dimethylcytidine, 5-(2-hydroxyethyl)cytidine, 5-chlorocytidine, 5-bromocytidine, N4-methyl-N4-aminocytidine, 5-aminocytidine, 5-nitrosocytidine, 5-(hydroxyalkyl)-cytidine, 5-(thioalkyl)-cytidine and cytidine glycol, 5-hydroxyuridine, 3-hydroxyethyluridine, 3-methyluridine, 02-methyluridine, 02-ethyluridine, 5-aminouridine, 04-methyluridine, 04-ethyluridine, 04-isobutyluridine, 04-alkyluridine, 5-nitrosouridine, 5-(hydroxyalkyl)-uridine, and 5-(thioalkyl)-uridine, 1,N6-ethenoadenosine, 3-methyladenosine, and N6-methyladenosine, 8-hydroxyguanosine, 06-methylguanosine, 06-ethylguanosine, 06-isopropylguanosine, 3,N2-ethenoguanosine, 06-alkylguanosine, 8-oxo-guanosine, 2,N3-ethenoguanosine, and 8-aminoguanosineas well as derivatives/analogues thereof. Examples of suitable nucleoside precursors, and synthesis thereof, are described in further detail in U.S. Patent Publication No. 20030119764, herein incorporated by reference in its entirety. Generally, these agents are added to the replication or transcription reaction thereby mutating the sequence. Intercalating agents such as proflavine, acriflavine, quinacrine and the like can also be used. The use of one or more chemical mutagens will allow for the generation of a wide array of nucleic acid alterations (such as but not limited to expansions or deletions of DNA segments within the context of a gene's coding region, a gene's intronic regions, or 5′ or 3′ proximal and/or distal regions, point mutations, altered repetitive sequences). In some embodiments, the chemical mutagen can be selected from the group consisting of 3-Chloro-4-(dichloromethyl)-5-hydroxy-2(5H)-furanone (MX) (CAS no. 77439-76-0), O,O-dimethyl-S-(phthalimidomethyl)phosphorodithioate (phos-met) (CAS no. 732-11-6), formaldehyde (CAS no. 50-00-0), 2-(2-furyl)-3-(5-nitro-2-furyl)acrylamide (AF-2) (CAS no. 3688-53-7), glyoxal (CAS no. 107-22-2), 6-mercaptopurine (CAS no. 50-44-2), N-(trichloromethylthio)-4-cyclohexane-1,2-dicarboximide(captan) (CAS no. 133-06-2), 2-aminopurine (CAS no. 452-06-2), methyl methane sulfonate (MMS) (CAS No. 66-27-3), 4-nitroquinoline 1-oxide (4-NQO) (CAS No. 56-57-5), N4-Aminocytidine (CAS no. 57294-74-3), sodium azide (CAS no. 26628-22-8), N-ethyl-N-nitrosourea (ENU) (CAS no. 759-73-9), N-methyl-N-nitrosourea (MNU) (CAS no. 820-60-0), 5-azacytidine (CAS no. 320-67-2), cumene hydroperoxide (CHP) (CAS no. 80-15-9), ethyl methanesulfonate (EMS) (CAS no. 62-50-0), N-ethyl-N-nitro-N-nitrosoguanidine (ENNG) (CAS no. 4245-77-6), N-methyl-N-nitro-N-nitrosoguanidine (MNNG) (CAS no. 70-25-7), 5-diazouracil (CAS no. 2435-76-9) and t-butyl hydroperoxide (BHP) (CAS no. 75-91-2).

Following long-standing patent law convention, the terms “a”, “an”, and “the” refer to “one or more” when used herein, including in the claims.

As used herein, the term “about”, when referring to a value or an amount, for example, relative to another measure, is meant to encompass variations of in some embodiments ±20%, in some embodiments ±10%, in some embodiments ±5%, in some embodiments ±1%, and in some embodiments ±0.1% from the specified value or amount, as such variations are appropriate. The term “about” can be applied to all values set forth herein.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “and/or” when used in the context of a listing of entities, refers to the entities being present singly or in combination. Thus, for example, the phrase “A, B, C, and/or D” includes A, B, C, and D individually, but also includes any and all combinations and sub-combinations of A, B, C, and D.

The term “comprising”, which is synonymous with “including,” “containing,” or “characterized by” is inclusive or open-ended and does not exclude additional, unrecited elements or method steps. “Comprising” is a term of art used in claim language which means that the named elements are present, but other elements can be added and still form a construct or method within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, “significance” or “significant” relates to a statistical analysis of the probability that there is a non-random association between two or more entities. To determine whether or not a relationship is “significant” or has “significance”, statistical manipulations of the data can be performed to calculate a probability, expressed in some embodiments as a “p-value”. Those p-values that fall below a user-defined cutoff point are regarded as significant. In some embodiments, a p-value less than or equal to 0.05, in some embodiments less than 0.01, in some embodiments less than 0.005, and in some embodiments less than 0.001, are regarded as significant.

As used herein, an “instructional material” includes a publication, a recording, a diagram, or any other medium of expression, which can be used to communicate the usefulness of the methods and reagents of the presently disclosed subject matter in a kit for directed evolution as described herein. Optionally, or alternately, the instructional material may describe one or more methods for directed evolution. The instructional material of the kit of the presently disclosed subject matter may, for example, be affixed to a container, which contains one or more reagents of the presently disclosed subject matter, or be shipped together with a container, which contains the reagents. Alternatively, the instructional material can be shipped separately from the container with the intention that the instructional material and the reagents be used cooperatively by the recipient.

### II. GENERAL CONSIDERATIONS

Synthetic biology and metabolic engineering aim to endow organisms with novel phenotypes through genetic engineering. Success in this endeavor often requires that an organism's genetic pathways (native or heterologous) be optimized to fit the application of interest. Such optimizations might include alterations to protein expression levels, secretion rates, thermostability, catalytic rates, binding constants, and specificity. With a good understanding of sequence-function relationships for the protein of interest, these optimization tasks can be readily performed with the aid of computers, or as a hybrid approach in which residues which are predicted to be important are preferentially mutated. However, predictive biochemical models do not exist for many proteins, especially those from non-model organisms and the growing body of genetic “dark matter” being found in microbial metagenomes. In these instances, random mutagenesis followed by screening remains the most effective approach to improve function.

Traditionally, directed evolution entails a series of laborious and costly steps, which include molecular cloning and transformation, which conspire to limit the throughput of directed evolution projects. In response, several continuous directed evolution methods have been developed to overcome these challenges. These include: Phage-Assisted Continuous Evolution (PACE). In this approach, the DNA sequence of interest is encoded on a phagemid from a filamentous phage (M13) such that improvements in phenotype correspond to increased expression of an essential phage gene, thereby improving phage production. This approach is limited to short (<5 kb) gene segments due to the packaging limitations of M13, and it is also limited to activities that can be coupled to expression, such as promoters, transcription factors, and RNA polymerases. Generally, DNA segments which increase the growth rate of bacteria cannot be engineered with this approach.

In vivo continuous evolution (ICE) uses a retrotransposon to mutate pathways up to 10 kb in length in yeast. This method is not applicable to bacteria and carries with it the possibility of background genomic adaptations.

OrthoRep implements an extrachromosomal plasmid to mutate large (<20 kb) segments of DNA in yeast. This method is not applicable to bacteria and carries with it the possibility of background genomic adaptations.

### III. REPRESENTATIVE EMBODIMENTS

The presently disclosed subject matter relates to an approach referred to as Autonomous Inducible Directed Evolution (AIDE; FIGS. 1, 6A, and 6C), which addresses challenges other methods do not address, which primarily include the ability to evolve large DNA segments, decoupling screening and mutagenesis steps, and avoiding the accumulation of off-target genomic mutations.

### FIG. 1. AIDE Overview

Step 1. Pathway of interest is assembled in a phagemid.
Step 2. (Upper left corner). Phagemid transformed to a bacterium containing a phage (e.g. P1) and a mutagenesis plasmid.
Step 3. (Top arrow #1) Mutagenesis in induced with an inducer (e.g. anhydrotetracycline) for 8-16 hours.
Step 4. (Top arrow #2) Phage lytic cycle is induced with an inducer (e.g. arabinose) for phagemid packaging.
Step 5. (Right arrow) WT strain or strain carrying phage and mutagenesis plasmids is infected with phage lysate.
Step 6. (Bottom arrow) Characterization and Analysis of evolved library, and potentially initiation of another AIDE cycle.

An aspect of the presently disclosed subject matter is the inclusion of an inducible lysis cycle in AIDE. Inducible lysis allows AIDE to accumulate more or less mutations in one round of evolution, vary the length of time over which beneficial phenotypes can develop, and decouple beneficial mutations on the phagemid from “hitchhiker” or “off target” mutations in the genome.

In some embodiments, the presently disclosed subject matter provides an engineered temperate phage, such as a P1 phage. In some embodiments, P1kc is an engineered variant of the P1 bacteriophage that has been optimized for efficient transduction. The components of this phage that have been modified are: 1) insertion of “stuffer” sequences to increase its genome size and 2) deletion of coi. The lysogenic and lytic cycles are controlled by the activity of Coi and C1. Coi is an inhibitor for C1. When coi is expressed, it blocks C1 activity, switching P1 to a lytic cycle. P1kc contains 10 kb of yeast genomic DNA added to the P1 genome. Yeast DNA is not expected to be expressed in E. coli. We used this P1kc to increase phagemid packaging rate. We found that P1kc reduces P1 phage packaging rate, thereby increasing phagemid packaging.

In some embodiments, a P1kc was constructed with the coi gene knocked out (P1kcAcoi). We quantitatively tested phagemid packaging rate in this system and noticed an increase in packaging/infection rates relative to P1kc.

Several advantages are provided by engineering a temperate phage, such as a P1 phage, versus another type of phage. For example, a temperate phage, such as P1, can package and transfer a large genome (˜90 kb) in phage particles, allowing us to evolve large DNA pathways using AIDE, much larger than can be achieved by phage-assisted continuous evolution (PACE, <5 kb) or more traditional error-prone PCR approaches (for which polymerase processivity and cloning efficiencies become limiting at ˜10 kb). Also, P1 is a temperate bacteriophage, meaning it can switch between lysogeny (stably replicating in a host cell) and lysis (making many copies of itself and bursting open its host cell). This exactly matches the requirements of directed evolution, where sometimes it can be desirable to allow time for a phenotype to develop, and other times it can be desirable to transfer the evolving DNA to a fresh host to eliminate off-target mutations. Further, P1 is known to infect multiple gram-negative bacteria, including Escherichia sp. and Klebsiella sp. This provides for the application of this approach in many bacterial species. AIDE can thus be implemented in different gram-negative bacteria such as Klebsiella pneumioniae.

In some embodiments, a phagemid is provided. In some embodiments, the phagemid is a P1 phagemid. In some embodiments, the phagemid comprises inducible components, additional phage components, components for engineering specific pathways, and combinations thereof. In some embodiments, the phagemid contains a system for turning on the lytic cycle of P1kc. In some embodiments, this comprises the coi gene under the control of an arabinose-inducible promoter. In some embodiments, the ribosome-binding site of the coi gene is modified.

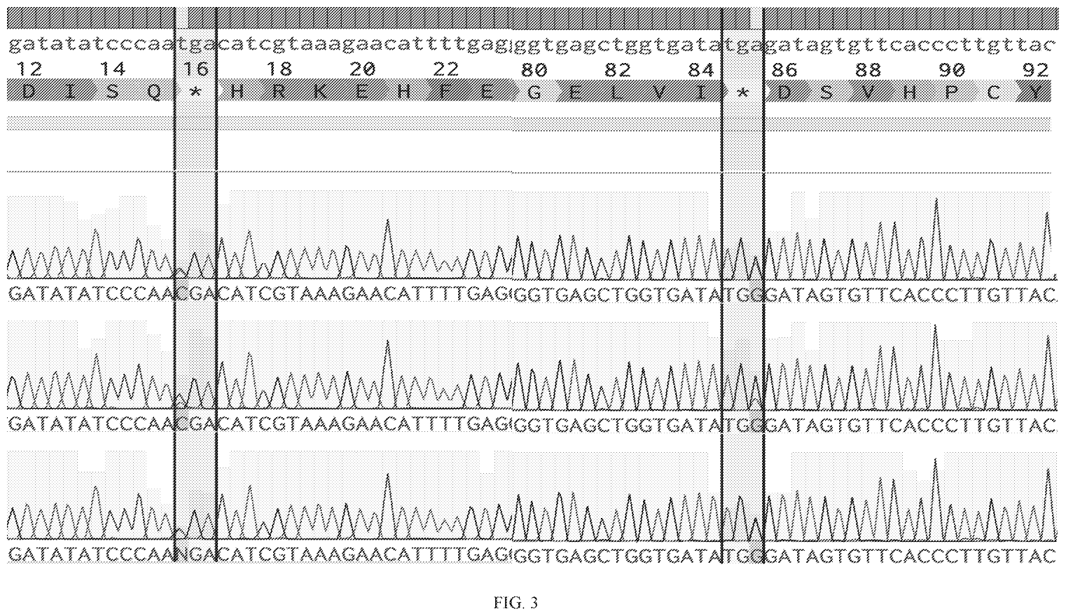

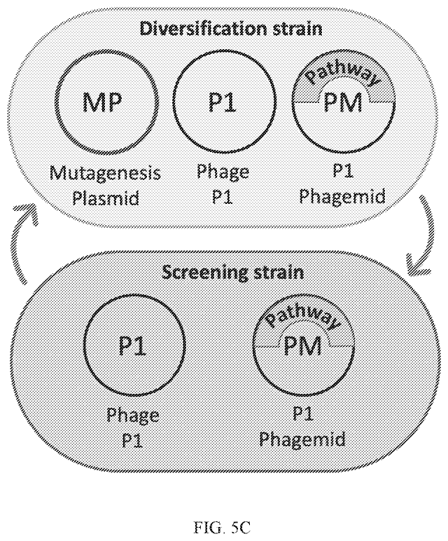

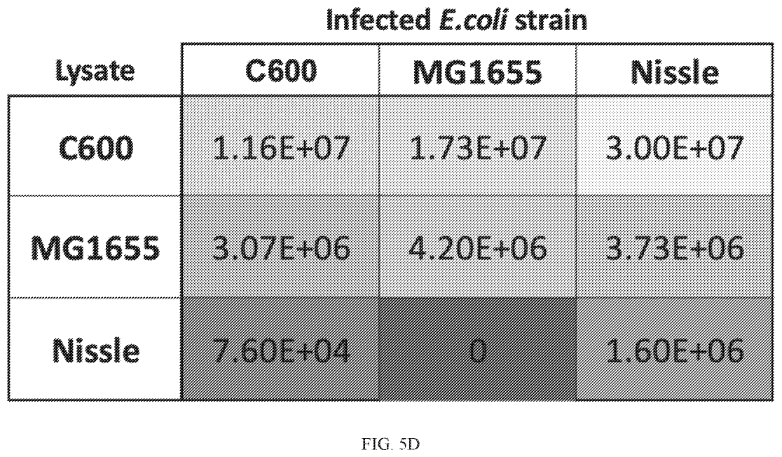

In AIDE, this phagemid is also modified to include the pathway to be mutated, i.e., the target gene sequence. Others have added DNA cargo to the P1 phagemid, but not for evolution purposes. As an example of an evolved pathway, FIGS. 3 and 5B shows AIDE's ability to revert a stop codon in antibiotic resistance genes. Chloramphenicol resistance genes with single stop codons introduced in the phagemid, and one AIDE cycle was performed, generating many strains in which mutations had converted this stop codon to a functional coding sequence. Thus, in some embodiments, the phagemid comprises a coi gene under the control of an arabinose-inducible promoter; a potentially modified ribosome binding site for the coi gene; and/or a pathway to be evolved.

Components of the phagemid can include any suitable component as would be apparent to one of ordinary skill in the art upon a review of the instant disclosure. Representative components include but are not limited to components to allow for targeted delivery. Representative components also include:
coi ribosome binding site: we have explored increasing the ribosome binding site strength of the coi gene. These experiments did not yield improvements when the system was tested in E. coli Nissle. As described below, however, E. coli C600 is another exemplary strain for performing AIDE in which ribosome binding site modifications in C600 are tested. Thus, in some embodiments, the ribosome binding site (RBS) of the Coi gene that is controlled by arabinose inducible system is altered. Altering the RBS of the Coi gene can tune C1 master repressor blocking and therefore the lytic cycle.
Toxin-antitoxin systems. doc and Phd are examples of a toxin-antitoxin system. Phd prevents cell death when P1 is in the cells and doc is the toxin gene, causing cell death in the absence of PhD. These two genes could be used to increase AIDE efficiency by clearing cells that do not carry P1.
P1 packaging sites (e.g. PacA and PacB): These are the sequences where P1 DNA is cleaved for packaging. Adding PacA and PacB sequences to phagemid can possibly increase phagemid packaging rates.
Cre: Cre system is cyclization recombinase. This can possibly increase phagemid survival in infected cells by stably cyclizing the phagemid after infection.

In some embodiments, a mutagenesis plasmid is provided. The mutagenesis plasmid can comprise any suitable component as would be apparent to one of ordinary skill in the art upon a review of the instant disclosure. Representative components include but are not limited to:
Antibiotic resistance gene: We switched MP6's normal chloramphenicol resistance gene to the kanamycin resistance gene so that we can maintain all 3 plasmiids in this system.
Mutagenesis induction system: We have explored both IPTG- and anhydrotetracycline-inducible systems. MP6 natively contains an arabinose-inducible system, which would conflict with the phage induction system. We found that IPTG induction does not work well in E. coli C600 because this strain lacks the appropriate transporter proteins (-lacY1, deletion of lactose permease (M protein)). However, this system can work in E. coli Nissle since it has the transporter. However, we found that this system is relatively leaky (i.e. exhibits expression even in the absence of inducer). We found that the tetracycline-inducible system has a very tight off state, which makes it suitable to induce mutagenesis in either C600 or Nissle. In some embodiments, ribosome binding sites for the genes in the mutagenesis operon are altered.

In some embodiments, the mutagenesis plasmid comprises components that confer mutagenesis. Representative fragments include but are not limited to an inducible system—such as one comprising a ligand-responsive repressor protein and a cognate promoter sequence and mutagenesis operon components (e.g., dnaq926, dam, seqA, emrR, UGI and cdaI).

Representative processes/steps used during Autonomous Inducible Directed Evolution (AIDE) are shown in FIGS. 1, 6A, and 6C. An inducible lysis cycle is an aspect of AIDE Inducible lysis allows AIDE to accumulate more or less mutations in one round of evolution, vary the length of time over which beneficial phenotypes can develop, and decouple beneficial mutations on the phagemid from “hitchhiker” or “off-target” mutations in the genome. All previously-developed directed evolution strategies can only evolve up 10 kb pathways. In some embodiments, AIDE relies on P1 to address this challenge, which allows packaging of large pathways and transforming them efficiently to new recipient cells. Other representative phage include lambda phage (canonically infects E. coli), Phages B025, 13035, B3056, PSA (which infect Listeria, Kilcher et al., PNAS Jan. 16, 2018 115 (3) 567-572; first published Jan. 3, 2018), and phages A2, FSW, and AT3 (which infect Lactobacillus). Upon a review of the instant disclosure, one of ordinary skill in the art could also consider art-recognized databases of phage, or methods of recovering temperate phage from nature, to identify a phage to which to apply AIDE.

In some embodiments, all mutagenesis, packaging, and infection steps are performed by the microbe or phage itself, thereby providing for the autonomous aspect of the presently disclosed subject matter. In some embodiments, these steps employ addition of inducer molecules, but this effort is very low compared to the labor required in current methods, such as PCR for mutagenesis or transformation for infection, Other approaches that provide for the autonomous aspect of the presently disclosed subject matter include but are not limited to robotic culture and addition of reagents in response to certain conditions. For example, robots can induce phage lysis, isolate phage particles, apply them to naive cells, culture these cells under selective conditions, and induce mutagenesis again upon reaching a certain cell density threshold. In some embodiments, within-microbe sensors which turn on mutagenesis or phage production in response to certain conditions, are provided. For example, the promoter controlling the mutagenesis plasmid can be swapped for a cell density-inducible promoter (e.g. enabled by quorum sensing), and the promoter controlling phage production can be swapped for a promoter which turns on a certain length of time after the cell density-inducible promoter turns on.

With reference to FIGS. 1, 6A, and 6C as non-limiting schematic presentations, representative, non-limiting examples of the presently disclosed subject matter comprise the following steps to provide for inductions of directed evolution. (1) Clone pathway of interest into phagemid. (2) Transform phage genome, phagemid, and mutagenesis plasmid into strain of interest. (3) Grow strain of interest (such as overnight, grow strain of interest (for example, containing: P1, phagemid and ISA265) up to saturation in rich media such as LB+0.5% glucose or Davis media); growth could be 1 h to 48 h or longer, depending on the growth rate of the strain). (4) Dilute cell culture to a desired dilution, such as 1000×, 250-2000×, and/or 2× to over 1000000×. (5) Add inducer of mutagenesis, and culture in a suitable media, such as LB media, LB media including appropriate antibiotics (kanamycin and chloramphenicol), any rich media, such as Davis media mentioned above, and also Tryptic Soy Broth or blood media), for a suitable length of time (such as 4 h, 4-16 hours, and/or 1 h to 48 h or longer. (6) Add inducer of phage lysis and packaging and incubate for a suitable length of time (such as 3 h, 90-180 minutes, and/or 1 h to 48 h. (7) Isolate phage particles, such as by addition of chloroform, centrifuging, and isolating supernatant and/or by a detergent or a bacteriolytic antibiotic, or a toxin protein, as an aspect of the isolation is the killing of residual cells). (8) Apply supernatant to wild-type cells, such as at OD=1 for 45 minutes, 0.8-1 OD and for 30-60 minutes, and/or 0.01 to 10 OD, 1 h to 48 h. (9) Add a quenching reagent to stop phagemid particles co-infection, such 200 mg/ml of Sodium citrate in Super Optimal Broth (SOB). As a non-limiting example, 200 mg/ml of Sodium citrate in Super Optimal Broth (SOB) stop co-infections and allow the cells to express the resistance gene to survive in the selective media. (10) Perform screen or selection on infected cells. (11) Cells that pass screen are returned to step 4 in the protocol. Steps 5-7 of above protocol are involved in elimination of off-target mutations.

Representative reagents for AIDE include but are not limited to Mutagenesis plasmids, Phagemids, phage genome, LB (Luria-Bertani) media, PLM (Phage Lysate Media), antibiotics, Arabinose, anhydrotetracycline, IPTG, chloroform and agar. These reagents can be provided in a kit in accordance with the presently disclosed subject matter.

Any desired pathways/enzymes/etc. as would be apparent to one of ordinary skill in the art upon a review of the instant disclosure can be targeted in accordance with the presently disclosed subject matter. For a desired pathway/enzyme/etc. a screen for activity is devised. The following list is a non-exhaustive list of representative, non-limiting examples: Genes encoding non-coding RNAs; Nutrition utilization pathways; Stress-responses genes; Transcription factors; DNA repair and replication enzymes; Metabolic pathways, including secondary metabolite biosynthetic gene clusters and central carbon utilization pathways; Genes required for colonization of host-associated and environmental habitats (termed “colonization factors); CRISPR/Cas systems, including Entire CRISPR/Cas systems; Phage tail fiber proteins or other phage structural components; and/or combinations of the above.

In some embodiments, the presently disclosed subject matter employs engineered microbes, such as but not limited to bacteria. The engineered microbes, such as engineered bacteria can comprise any suitable modification as would be apparent to one of ordinary skill in the art upon a review of the instant disclosure. In some embodiments, E. coli C600, which is a historical strain that has been used for P1 phage cycles, is employed. Other strains can be used with P1 without modifications, such as Klebsiella strains mentioned above, or E. coli Nissle mentioned above. Other strains are engineered for use as P1 hosts by making the following changes (via genome editing or incorporating genes into the phagemid itself) to make them more “C600-like”: F−=Does not carry the F plasmid; glnV44=suppression of amber (UAG) stop codons by insertion of glutamine; required for growth of some phage; and λ−=lambda lysogen deletion.

Any desired products of AIDE as would be apparent to one of ordinary skill in the art upon a review of the instant disclosure can be prepared in accordance with the presently disclosed subject matter. The following list is a non-exhaustive list of representative, non-limiting examples: Probiotics (for humans, animals, or plants); Strains that produce value-added compounds (biologic drugs, small-molecule drugs, other small-molecule chemicals, biofuels); Bioremediation strains.

The following list is a non-exhaustive list of representative, non-limiting examples. Engineered probiotics can be used to: colonize their host (humans, animals, plants) for defined lengths of time before exiting, deliver drugs/vitamins/nutrients to their host, degrade toxic molecules, inhibit/enhance the growth of surrounding microbes via production of toxins, signaling molecules, or nutrients, and/or deliver nucleic acids or proteins to host cells.

Engineered bioproduction strains can be used to produce: biologic drugs, such as but not limited to small-molecule drugs, other small-molecule chemicals, polymers, and biofuels; bioremediation strains can be used to degrade or sequester, potentially in a bioproduction or probiotic context, heavy metals, radioactive substances, toxins, plastic, rubber, petroleum, food waste, agricultural residues (plant or animal); and waste gases (SO₂, SO₃, NO₂, NO₃, CO, CO₂).

It is believed that the presently disclosed subject matter provides for the first time the use of P1 for mutagenesis and screening. Prior efforts have only used P1 for delivery of DNA cargo. In these prior efforts, the goal was to maximize P1 packaging and delivery. However, an aspect of the presently disclosed subject matter is to minimize P1 packaging, thereby increasing the relative packaging of phagemid. In general, no temperate phage has ever been used to perform directed evolution. In representative embodiments, particular MgCl₂ and CaCl₂) concentrations were identified that enhance infection rate. 100 mM MgCl₂ and 5 mM CaCl₂) are commonly added to LB media (forming phage lysate medium (PLM)) in studies of P1 phage (Kittleson, Joshua T., et al. ACS Synth. Biol. 1, 583-589 (2012)). We found that when the concentration of both salts in PLM is increased by 40% (140 mM MgCl2 and 7 mM CaCl2)), the number of PM-containing cells increased 2.2-fold to 1.3*10\hat{ }6. We called the new medium ePLM. Particular ratios of phage lysate to cells, and cell growth phase, are also provided which enhance and even maximize infection rate relative to previously reported protocols. We found that the ratio (lysate from 5*10\hat{ }8 cells applied to 3*10\hat{ }8 cells, defined as a ratio of 1:1) was past its saturation level, with reduced amounts of lysate providing similar values.

The presently disclosed subject matter performs directed evolution on the largest pathway sizes; it can easily decouple mutations in the pathway from genomic hitchhiker or off-target mutations; it can stop mutagenesis for any length of time while the phenotype of interest is being measured; temperate phages exist for many bacteria, and the regulators of their lifecycle (analogous to coi—e.g. cI and cro for lambda phage (Savageau, Michael A. Handbook of Systems Biology (pp. 287-310) (2013)) and recA (De Paepe, Marianne, et al. PLoS Genet 10.3: e1004181 (2014))) are known, indicating that the presently disclosed subject matter is applicable to many different microbes, including but not limited to many different bacterial species.

In some embodiments of the presently disclosed subject matter, the DNA to be evolved (the “cargo”) is encoded within a DNA sequence (“evolution cassette”, or EC) that can be loaded in phage particles. EC is delivered to a bacterium. The bacterium also contains a DNA sequence (“replication cassette”, or RC) that produces EC-bearing phage in response to an external signal. Mutations are introduced to EC. Then, cells containing EC are screened (to identify those with favorable properties) and/or EC is transferred to another bacterium. Transfer occurs by providing an external signal that directs a RC-containing bacterium to load PM into phage particles. These phage particles are incubated with another bacterium to complete the transfer. Screening and transfer can occur in any order, and any number of times, before mutations are introduced to EC again.

We have used E. coli to demonstrate this method, but those skilled in the art will recognize that any organism containing the elements above would be suitable for the method.

In some embodiments, we have used a plasmid that encodes genes that introduce mutations to EC in response to an external signal (“mutagenesis cassette”, or MC). This plasmid has been modified from its original description (Badran, A. H. and Liu, D. R., 2015, Nature communications, 6, 8425) to be inducible by anhydrotetracycline, rather than arabinose. However, those skilled in the art will recognize that any mutagenesis technique, such as a physical (e.g. UV light), chemical (e.g. EMS), or enzymatic (e.g. CRISPR/Cas-based) mutagen will work.

In some embodiments, we have used a modified version of the E. coli phage P1 to package the cargo (i.e. RC), that has been engineered by others (Kittleson, Joshua T., et al., 2012, ACS synthetic biology, 1(12), 583-589) to be inducible in response to arabinose. Though those skilled in the art will recognize that any sequence that directs the production of cargo-containing particles in response to an external signal will work for this method. Non-limiting examples include other temperate bacteriophages such as lambda phage.

In some embodiments, we have used a common P1 phagemid (phagemid, J72103) as our EC sequence, though those skilled in the art will recognize that any sequence that can be packaged by RC will be suitable.

Cargo beyond 85 kb in size can be used in AIDE in several ways:
1) Using the same RC (e.g., P1), separating the cargo across multiple ECs, and infecting cells with each one. These infections can occur simultaneously or sequentially. All other steps are the same as described elsewhere herein.
2) Choosing RC and EC pairs that support cargo beyond 85 kb in size. Temperate phages (other than P1) can be hundreds of kb in size, and their lysis regulators can be similarly engineered as we have performed here for P1 to respond to external signals and be compatible with AIDE.
3) Using multiple different, independently-controllable RC/EC pairs.

CRISPR/Cas nucleases, nickases, base editors, transposons, transcriptional activators, etc can be improved using AIDE by placing their coding sequences in EC. On- and off-target sites can be placed on a separate plasmid or in the genome. (On-target sites can be designed to yield fluorescence or antibiotic resistance if the CRISPR/Cas system exhibits the desired activity. Off-target sites can be designed to yield a different fluorescence or express a counterselectable marker if the CRISPR/Cas system is active at that site. AIDE can proceed by mutating EC, then inducing RC to move EC to fresh cells, followed by screening those cells for the desired fluorescence/resistance properties. EC-can be re-mutated in successful cells, and the cycle can repeat.

The ability of phage to infect cells and deliver DNA can be improved using AIDE by first engineering the phage of interest to replicate in response to an external signal, as we have performed here for the P1 phage. This new engineered phage becomes RC. Since RC can package itself, mutations can be introduced to RC, RC-containing phage can be produced, and those phages exhibiting the desired infection properties (e.g. infecting the desired bacteria or avoiding undesired bacteria) are recovered. Mutations can be re-induced in these phages for additional cycles of infection. If it is desired for the phage to deliver a DNA cargo (e.g. a CRISPR nuclease or drug production pathway), then the cargo can be added to RC or EC, as desired, and mutagenesis can be induced. Mutated RC/EC can be applied to the target bacteria, and those bacteria exhibiting the desired activity (e.g. nuclease activity or drug production) can be recovered. EC/RC can then be subjected to additional rounds of selection.

Microbial growth rates can be increased using AIDE by placing genes of interest on EC, mutating them, and screening the resulting cells for increased abundance under the condition of interest. This type of experiment includes engineering cells to be more tolerant to inhibitors, such as alcohols, acids, bases, high or low temperatures, desiccation, radiation, high or low pressure, and high or low osmolyte concentration. These types of experiments also include engineering for increased survival in challenging environments, for example in the human or animal gastrointestinal tract, on human or animal skin, on surfaces, in long-term storage, in plant tissue, on plant roots, in the soil, or in bioreactors. These types of experiments also include engineering phenotypes such as increased/decreased adherence to particular materials or increased swim speed toward particular chemicals. Genes that may be placed within EC include multi-gene carbon utilization pathways, transcriptional regulators (that are specific to a few genes or that regulate many genes), chaperone proteins, proteins involved in protein secretion, and proteins involved in cell membrane/wall biosynthesis. Importantly, due to the large allowable size of EC, many genes can be evolved at the same time.

Bacterial sensors can be developed using AIDE by placing candidate sensor proteins (e.g. two-component systems or histidine kinases) on EC, mutating them, and screening the resulting cells for responsiveness and specificity to the desired compound. For example, after mutagenesis, EC may be transferred to cells in which the candidate sensor protein is coupled to the production of GFP or an antibiotic resistance gene. Only cells exhibiting responsiveness to the desired analyte will be recovered after selection. Also, after mutagenesis, EC may be transferred to cells in which the candidate sensor protein is coupled to the production of a fluorescent gene or counter-selectable marker, such as sacB. Cells which exhibit responsiveness to off-target compounds may be removed in this way.

The ability of cells to produce desired molecules may be engineered using AIDE by putting genes involved in molecule synthesis on EC. Such genes include the synthesis pathway itself, and/or auxiliary genes such as chaperone proteins, sigma factors, and cell wall/membrane synthesis proteins. Once mutated, EC can be transferred into a cell containing a biosensor for the desired molecule (see above), coupled to GFP or antibiotic resistance expression. Those cells producing the desired molecule will exhibit higher fluorescence or growth rates in selective media and can be readily recovered. In successful cells, RC will be induced to package EC, and EC can be subjected to further rounds of mutagenesis and selection in fresh cells. Importantly, the ability of AIDE to easily mobilize EC between cells prevents them from “cheating” and mutating the biosensor during selection. This method is applicable to a wide range of molecules, including natural products, biofuels, feedstock molecules, vitamins, antibiotics, drug molecules, proteins, and enzymes.

### IV. EXAMPLES

The Examples provided herein have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example—Optimization of Phagemid Production and Infection Rates

We demonstrated AIDE using a modified version of the phage P1 that undergoes lysis in response to the addition of arabinose (P1kcΔcoi). This was achieved by knocking out coi, which encodes a repressor of c1, and placing it under the control of an arabinose-inducible promoter on a P1 phagemid (PM). In the uninduced state, c1 is expressed and maintains P1 lysogeny. After coi induction, c1 can no longer maintain lysogeny, resulting in P1 particle production and lysis. Inducible mutagenesis was achieved using a previously-described plasmid (MP6). We began by increasing P1 phagemid packaging and infection levels, as these metrics define the number of library members that can be passaged between evolutionary rounds and affect the explorable sequence space. Over the course of these optimization experiments, we were able to increase P1 transduction rates by more than 4,000 fold over previous methods (Kittleson, Joshua T., et al. ACS Synth. Biol. 1, 583-589 (2012), Kasman, L. M. et al. J. Virol. 76, 5557-5564 (2002)).

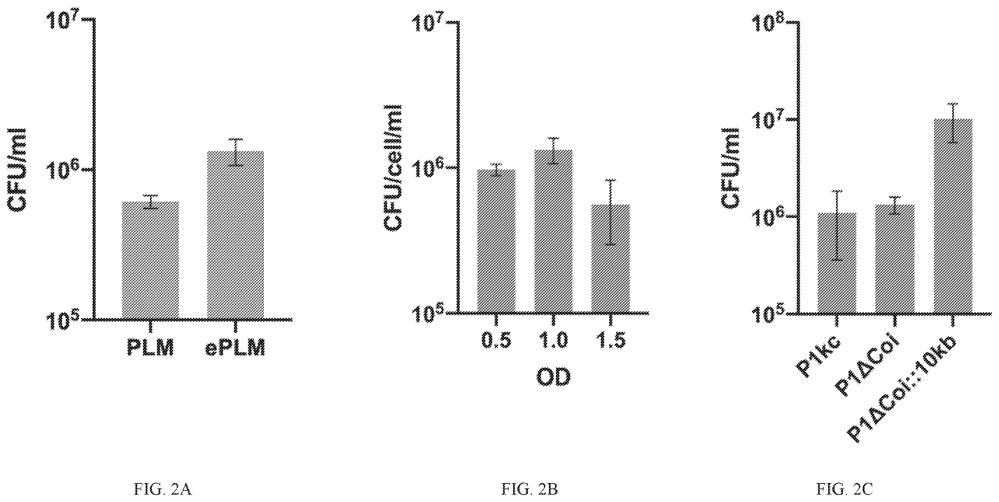

We first wished to increase P1 phagemid infection and packaging levels, as these metrics define the number of library members which can be passaged between evolutionary rounds. In these experiments, E. coli C600 containing P1kcΔcoi and 12 kbp phagemid cultures were grown to OD 1, had lysis induced, and 1 mL of the resulting lysate was applied to 1 mL of media containing 3×10⁸ recipient E. coli C600 cells containing P1kcΔcoi. The resulting mixture was plated on media selective for the phagemid to determine overall phage production and infection rates. First, we varied the optical density of the recipient cells. We found that cells grown to an optical density of 1.0 exhibited the highest phage infection rate, yielding over 10⁶ colony forming units per mL of infected cells on media selective for the phagemid (FIG. 2A).

Next, we investigated the composition of the media in which infection was performed. Salt composition is known to influence the physiological state and metabolic function of bacterial cells. In particular, MgCbh and CaCh help gram-negative bacteria stabilize negatively charged lipopolysaccharides on the membrane. Previous studies have shown that adding 100 mM MgCh and 5 mM CaCbh to LB media increases phage production and infection levels. This growth medium is called phage lysate medium (PLM). However, we hypothesized that increasing the concentration of MgCL and CaCl₂ in the growth medium can enhance P1 phage absorption rate and increase cell stability during phage production and infection. In a representative embodiment, we found that when the concentration of both salts in PLM is increased by 40%, the infection and packaging levels of phagemid increased by 2.2-fold. We called the new medium enhanced Phage Lysate Medium (ePLM) (FIG. 2B)

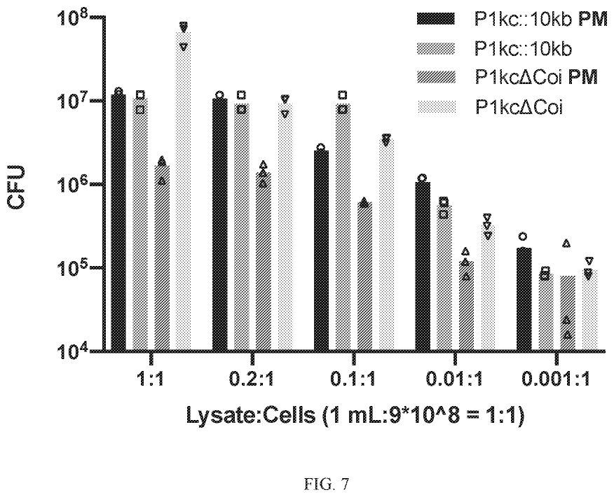

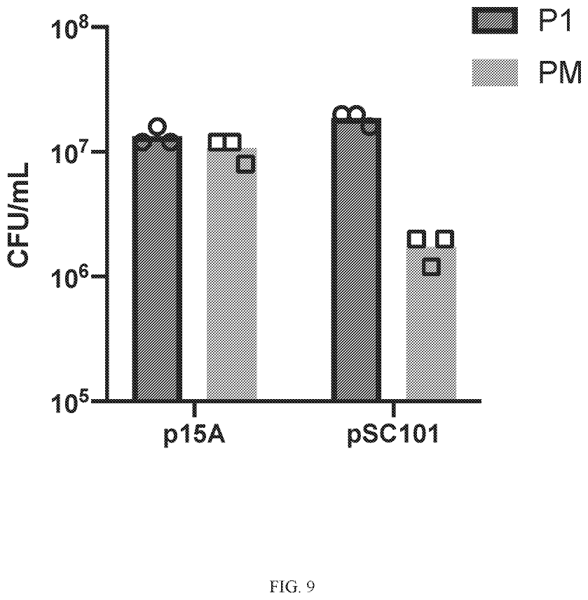

Next, we hypothesized that reducing the ability of P1kcΔcoi to be packaged in phagemids would increase the proportion of particles carrying PM. We found that inserting 10 kb of yeast DNA into P1kc (generating P1kc::10 kb) increased transferable library size by an additional 7.6-fold, to 1.2×10\hat{ }7 (FIGS. 2C, 7). Similarly, we found that PM packaging was copy-number dependent, with lower PM copy numbers resulting in a reduced number of transduced cells (FIG. 9).

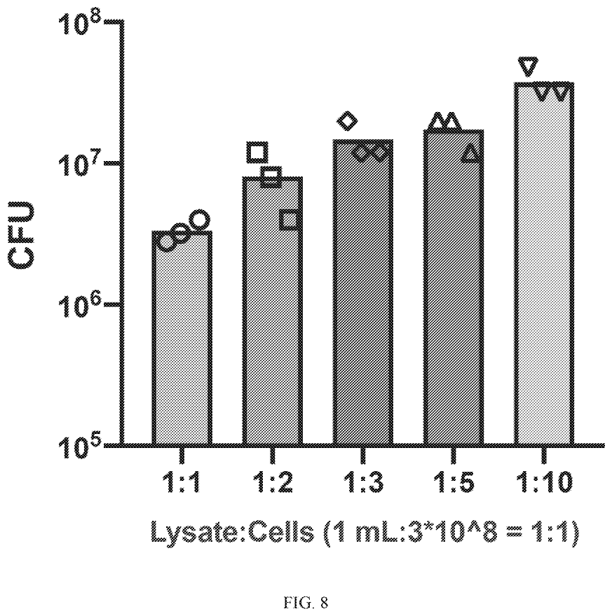

Although we expected that increasing the amount of lysate applied to naive cells would increase the number of transduced cells, we found that the ratio we had been using (lysate from 10\hat{ }8 cells applied to 3*10\hat{ }8 cells, defined as a ratio of 1:1) was past its saturation level, with reduced amounts of lysate providing similar values (FIG. 7). Therefore, we varied the number of naive cells, holding the lysate volume constant. As expected, we observed a linear relationship between the number of naive cells and the number of infected cells (FIG. 8), indicating that AIDE library sizes can be easily increased by scaling up lysate and cell amounts.

### Example—AIDE Enables Evolution of Large DNA Constructs

Because current directed evolution techniques are unable to generate mutations in DNA segments larger than 10 kb in size, we investigated the size-dependence of phagemid production and packaging using AIDE. We produced phage particles from cells containing phagemids which are 9 kb, 12 kb, 24 kb, and 42 kb in size, and infected wild-type E. coli cells with these particles. We found that phagemid size exerted a very minor effect on the number of infected cells (FIG. 5A). While we expect a substantial reduction of library size with phagemids larger than wild-type P1 (FIG. 2C), this indicates that AIDE is capable of efficiently evolving large multi-gene pathways.

### Example—AIDE Enables Gain-of-Function Mutations

Although the mutation rate of the mutagenesis plasmid we adapted for this work (MP6) was previously reported, switching the inducible system from its original arabinose-inducible system to an anhydrotetracycline (aTc)-inducible system (forming aTc-MP) or an IPTG-inducible system (forming IPTG-MP) and selection marker from the original chloramphenicol resistance to kanamycin resistance might change the mutation rate. We therefore explored both IPTG- and anhydrotetracycline-inducible systems because, while MP6 natively contains an arabinose-inducible system this would conflict with the phage induction system which is also induced by arabinose. We found that IPTG induction does not work well in E. coli C600 because this strain lacks the appropriate transporter proteins, for example lactose transporter proteins. However, we found that IPTG induction can work in E. coli Nissle (a probiotic) since it has the transporter, for example lactose transporter proteins. However, we found that IPTG induction is relatively leaky (i.e. exhibits expression even in the absence of inducer), making it problematic for directed evolution studies. In contrast, we found that the tetracycline-inducible system has a very tight off state, which makes it suitable to induce mutagenesis in either E. coli C600 or E. coli Nissle. To test the mutation rate conferred by aTc-MP, we generated phagemids containing chloramphenicol resistance gene (CmR) with either one or two premature stop codons. Random mutagenesis of CMR will yield variants with the stop codon(s) reverted to a functional codon. We found that induction of the mutagenesis plasmid enabled time-dependent increases to the number of CmR-resistant cells (from a starting pool of 10\hat{ }8 cells housing the CmR gene containing a single stop codon), supporting the notion that AIDE enables inducible mutagenesis of defined DNA cargo (FIG. 3). We additionally found that AIDE enables the simultaneous reversion of two premature stop codons in CmR at a rate of 3 per 10\hat{ }8 induced cells. The expected reversion mutations were confirmed via sequencing (FIG. 3). See also Table 4.

### Example—AIDE Enables Loss-of-Function Mutations

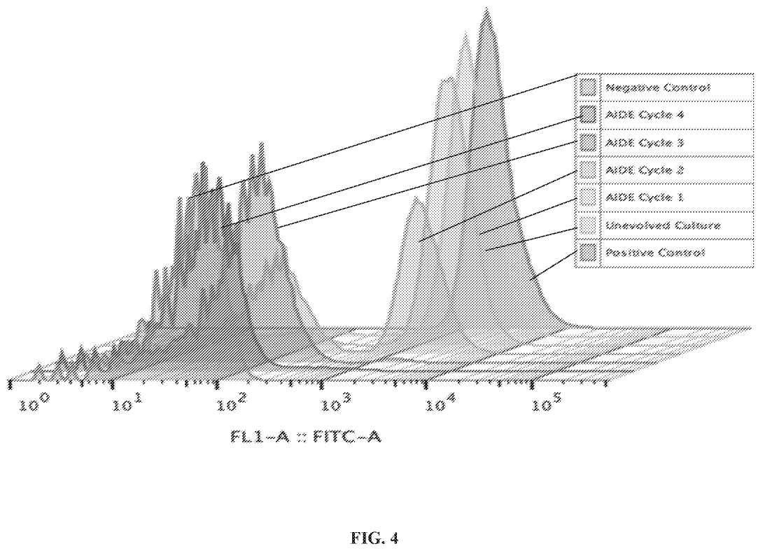

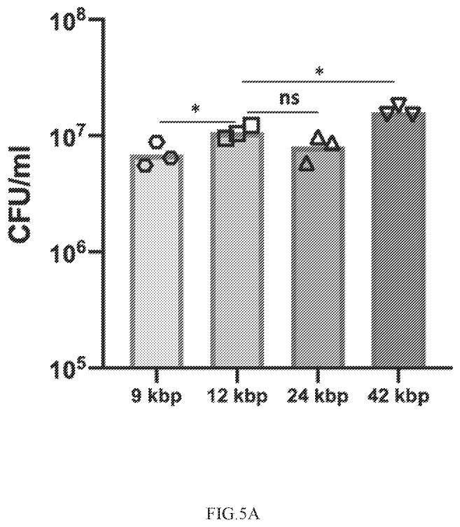

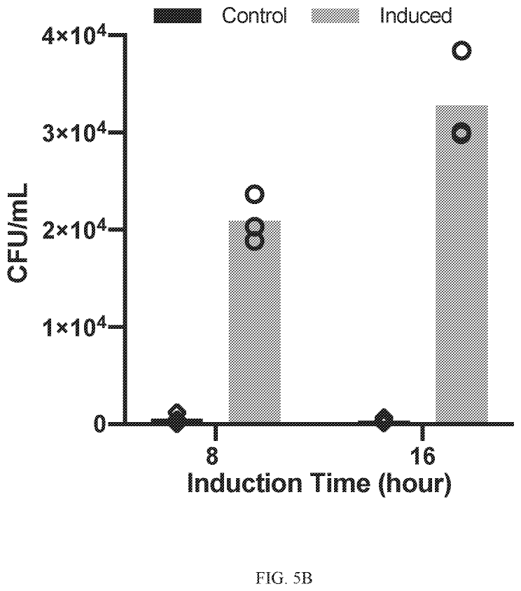

The above experiments showed that mutagenesis via aTc-MP enables reversion of stop codons present in resistance genes. However, a component of AIDE is transfer of these mutants to new cells. Loss-of-function provides a good readout of this capability, since individual loss-of-function variants are difficult to distinguish in cells containing many functional plasmids. Therefore, we placed a gene encoding green fluorescent protein (GFP) on the AIDE phagemid. We performed 4 total cycles of mutagenesis and passage to wild-type cells. We found that as the number of cycles increased, the number of phagemid-containing cells containing nonfunctional GFP variants increased. We hypothesize that GFP production instills a growth defect, leading to a selective pressure for reduced GFP expression. These experiments demonstrate mutagenesis of a target gene, followed by delivery of this mutated gene to target cells via phage. See FIG. 4.

### Step-by-Step of Increasing Cell Fitness by Introducing Random Recessive Mutations in p15A-sfGFP Phagemid Via AIDE

1. sfGFP phagemid (Addgene #40782) was miniprepped and transformed to ISA372 forming ISA384.
2. ISA384 was grown overnight in LB with 1% glucose and Kan and Cm at 37° C. and 250 rpm.
3. Cells were then harvested at 5000 rpm for 5 minutes and washed with 1×PBS.
4. Washed cells were inoculated into fresh LB media with Kan and Cm.
5. Mutagenesis was induced starting at OD 0.01 with 200 ng/mL aTc for 16 hours.
6. Evolved culture was sub-inoculated to OD 0.1 in ePLM at 37° C. and 250 rpm and grown OD 1.0.
7. 20% Arabinose (0.01 volumes) was added to induce Phage production for 2 hours.
8. Lysed cultures were transferred to 15 mL Eppendorf conical tubes
9. Chloroform ( 1/40 volume) was added to the reaction to kill residual cells.
10. Phage lysate was spun down at 5000 rpm for 10 minutes at 4° C.
11. Phage lysate was transferred (avoid cell pellet) to a new 15 mL Eppendorf tube.
12. Phage lysate can then be used directly or stored at 4° C. for 1 year and indefinitely at −80° C.
13. E. coli C600 strain containing P1kc:10 kb (ISA222) was grown overnight in LB media.
14. ISA222 was then inoculated to 30 mL ePLM (1:100 dilution) and grown at 37° C. and 250 rpm to OD 1.0.
15. The cells were then harvested at 5000 rpm for 5 minutes and resuspended in 10 mL ePLM.
16. 1 mL Phage lysate was used to infect 1 mL of ISA222 from step 15.
17. Infection reaction was inoculated into 100 mL LB+Cm for fitness selection. Uninfected cells were killed with Cm in this step.
18. Cells were grown to saturation and inoculated (1:200) into 100 mL fresh LB+Cm, 2^{nd} round of selection.
19. Screened culture was sub-inoculated for phage production (Steps 6-12).
20. Phage lysate produced after screening was then used to infect ISA372.
21. Infection reaction was inoculated into 25 mL LB with Cm and Kan. Uninfected cells were killed with Cm and in this step
22. Steps 2-21 were repeated 3 times for a total of 4 AIDE cycles.
23. Phage lysates produced from each cycle after selection with controls (no mutagenesis induced) was used to infect wild type E. coli C600.
24. Infection reactions were inoculated into 25 mL LB with Cm and grown overnight.
25. Grown cultures were inoculated into 96 well plates (1:100 dilution) and grown to OD 0.5 in plate reader.
26. Cells were then diluted with LB media and run in flow cytometry with negative (wild type cells) and positive (unevolved phagemid) controls.

TABLE 1
Detected recessive mutations in p15A-sfGFP
Phagemid after 4 AIDE cycles. All the
mutations were detected in sfGFP.
*stop codon.

| Amino Acid Change | Sequence |
|---|---|
| G33D | GGT -> GAC |
| P56L | CCT -> CTC |
| W57* | TGG -> TGA |
| T621 | ACC -> ATC |
| TY92* | TAT -> TAA |
| A110V | GCG -> GTC |
| G127D | GGC -> GAC |
| Q157* | CAA -> TAA |

### Example—Effects of Bacterial Strain on Packaging and Infection Rates

Different E. coli strains have different packaging and infection rates, AIDE was tested in E. coli C600, E. coli Nissle and E. coli MG1655. We quantitatively tested packaging and infection rates in E. coli Nissle and E. coli MG1655 and found that both strains have less packaging and infection rates relative to E. coli C600 (FIG. 5D). Thus, in some embodiments, E. coli C600 is selected as a host cell for P1 and therefore AIDE.

We measured the rates of packaging and infection in E. coli Nissle and found that they were not as high as in E. coli C600. We attempted to increase phagemid packaging rates by increasing the strength of the ribosome binding site controlling coi expression. We found that this did not improve phagemid packaging in E. coli Nissle.

### Example—Raffinose and Melezitose

Raffinose and Melezitose (potential prebiotics) utilization pathway: raffinose is a sugar that can be found certain vegetables and melezitose can be found in honey. Probiotic E. coli could be modified to consume raffinose and melezitose by evolving a 17-gene pathway from Bifidobacterium breve UCC2003. The goal of controlling probiotic residence time via dietary consumption of these sugars is similar to the below case study for raffinose.

### Example—E. coli Modifications

Several E. coli sigma factors are placed on the phagemid and evolution is performed on these sigma factors to adapt E. coli to overcome certain environmental stresses, including low pH (simulating the stomach, for example), gut transit, high temperature, and high alcohol concentrations. This strategy can also be used to obtain an E. coli strain with improved residence time in the mammalian gut.

### Example Biofuel Production—Directed Evolution of Tolerance to and Catabolism of Lignocellulosic Breakdown Products

In this Example, tolerance is engineered to growth inhibitors present in lignocellulosic feedstocks through global Transcriptional Machinery Engineering. These growth inhibitors include lignin breakdown products, high sugar concentrations, low pH, and high temperature. E. coli contains 7 sigma/anti-sigma pairs which collectively exert broad control over the cellular transcriptome. Mutating these transcription factors is expected to result in broad changes to the host transcriptome, thereby allowing evolution to improve E. coli's global regulatory network for improved tolerance. The evolvability of each protein alone is tested, as is a tolerance “super-cassette” containing all 14 proteins (SC), to test the hypothesis that increasing the number of sigma factors under selection increases adaptation rate. Experiments are performed on individual growth inhibitors and combinations thereof, culminating in an experiment evolving improved growth on crude pretreated biomass.

Next, carbon source utilization is engineered. Importing heterologous carbon source utilization pathways to a new host is often required to merge high biofuel production with high biomass consumption phenotypes, yet biomass consumption can remain low in the new host. AIDE enables facile adaptation of heterologous lignocellulose degradation pathways to a new host. Biomass degradation pathways from several proficient biomass-consumers are expressed in PM, and evolution proceeds with growth on complex carbohydrates as a selective pressure. Many features of these pathways (i.e. GC content, codon usage, stability, expression interactions with other proteins) likely are optimized to function desirably in E. coli and therefore provide good tests of the AIDE method. SC are included with the biomass degradation pathway to enable transcriptional rewiring in tandem with improved enzyme activity.

For each phenotype, strains are recovered and RNA-seq can be performed during growth on the substrate of interest in order to learn the cellular functions which are beneficial (i.e. overexpressed) or detrimental (reduced expression) to tolerance and utilization of biomass. Deep sequencing of evolved pathways can also be performed over evolutionary rounds to determine which components of the engineered pathways are rate-limiting (i.e. those that are mutated first or most extensively). It is expected that these analyses will yield novel systems-level insights to biomass utilization by E. coli.

### Example Biofuel Production—Directed Evolution of Tolerance to and Production of High Biofuel Titers

The known toxicity of many biofuel products to E. coli can be first addressed. To reveal the underlying basis of this toxicity and increase the tolerance of E. coli to these compounds, directed evolution of SC is performed as in the Example immediately above in increasing concentrations of these compounds. Transcriptomics and deep sequencing of evolved mutants are expected to reveal components of cellular machinery which limit growth in the presence of these molecules. For each phenotype, strains are recovered and RNA-seq can be performed during growth on the substrate of interest in order to learn the cellular functions which are beneficial (i.e. overexpressed) or detrimental (reduced expression) to tolerance and utilization of biomass.

Next, production of biofuel molecules is improved via two case studies coupling microbial growth and biofuel productivity. First, terpene production in E. coli is affected by the toxicity of the precursor farnesyl pyrophosphate (FPP), providing a selective pressure for pathway inactivation during fermentation.

It is expected that by improving the tolerance of E. coli to FPP, more flux can be directed toward terpene production. Therefore, SC can be evolved in a strain engineered to produce FPP. While traditional adaptive evolution experiments would be stymied by mutations to the FPP pathway (rather than improvements to tolerance), AIDE exploits periodic transfer to an un-evolved host in order to eliminate these “cheaters”. Second, E. coli strains have been produced which require on ethanol and isobutanol formation in order to grow. AIDE is performed in these strains incorporating the biofuel formation pathway and SC into PM, thereby making use of the large packaging capacity of a temperate phage like P1.

Collectively, these biofuel production Examples demonstrate a facile “system” directed evolution approach which improves the biofuel-producing capacity of E. coli, and yields new insights into the systems biology of biofuel production. This is because AIDE can optimize entire pathways (not just individual enzymes) and automatically reveals the rate-limiting node in a system via analysis of mutation order. Notably, phage with similar lifestyle control elements as P1 infect many bacterial taxa. The presently disclosed subject matter therefore provides for translation to other bacteria with relevance to biofuels production. Also, AIDE is easily amenable to automation in microwell plates—allowing high replication and many phenotypes to be engineered at once, thereby reducing cost of engineering. Due to its power and ease of use, it is envisioned that AIDE will allow the biofuels industry to quickly adapt production strains to new process conditions (e.g. temperatures, feedstocks, oxygen levels, reactor designs), and reveal numerous insights regarding the systems biology of complex biofuel production phenotypes.

### Example—Mutagenesis and Screening Steps are Decoupled in AIDE

Because mutagenesis and screening steps are decoupled in AIDE, we hypothesized that the strain that is used for screening does not have to be the same as the strain that is used for library generation (FIGS. 5A through 5D). This would be beneficial if the ideal screening strain has a limited phage production capacity. As examples, we found that phage lysate produced from 5*10\hat{ }8 E. coli C600 cells can passage >10\hat{ }7 variants to E. coli MG1655 and E. coli Nissle 1917 (FIG. 5D). On the other hand, MG1655 and Nissle can only passage 4.9×10\hat{ }6 and 6.9×10\hat{ }4 variants back to C600 (FIG. 5D), respectively. These results indicate that C600 is well-suited for production and packaging of large libraries, enabling these libraries to be screened in a more appropriate strain, for example incorporating biosensors or production-coupled growth circuits.

### Example—Evolving a Multi-Gene Phenotype

To demonstrate AIDE's capability to evolve a simplistic multi-gene phenotype, we assembled sfGFP on a phagemid containing the pSC101 origin. We chose the pSC101 origin because of its stringent Rep101-dependent replication mechanism and its low copy number (Furuno et al., J. Gen. Appl. Microbiol. 46, 29-37 (2000)). In this setting, GFP fluorescence is controlled by at least 4 different genetic elements (the GFP coding sequence and its promoter, as well as Rep101 and its promoter). We found that after two sequential rounds of mutagenesis and passage to fresh cells, (FIG. 6A), we were able to visually isolate seven (7) highly fluorescent colonies. Sequencing Rep101 and sfGFP yielded mutations exclusively in Rep101. To separate these mutations from unknown mutations potentially present in other parts of PM, we cloned these Rep101 variants into an unmutated PM-sfGFP vector and measured fluorescence via flow cytometry. All variants yielded significantly higher fluorescence than wild-type (FIG. 6B). Most of the Rep101 mutations present in these clones (R46W, M78I, E93G, E93K, K102E, and E115K) were previously found to increase the copy number of pSC101 origin (Thompson et al., Sci. Rep. 8:1590 (2018)), while one (I94N) was novel. See Table 2 below. It is therefore likely that the increase in GFP production in these isolates is due to an increased phagemid copy number. This result is reasonable because sfGFP has already been optimized for high stability and fluorescence in prior studies (Pédelacq, J., Cabantous, S., Tran, T. et al. Nat Biotechnol 24, 79-88 (2006)), and so increasing the copy number of the plasmid may be an easier path to achieve higher GFP expression.

### Step-by-Step of Improving pSC101-sfGFP Fluorescence Via AIDE

1. pSC101-sfGFP phagemid was cloned via Gibson assembly forming ISA410.
2. ISA 410 was transformed to ISA372 forming ISA426.
3. ISA426 was grown overnight in LB with 1% glucose and Kan and Cm at 37° C. and 250 rpm.
4. Cells were then harvested at 5000 rpm for 5 minutes and washed with 1×PBS.
5. Washed cells were inoculated into fresh LB media with Kan and Cm.
6. Mutagenesis was induced starting at OD 0.01 with 200 ng/mL aTc for 16 hours.
7. Evolved culture was sub-inoculated to OD 0.1 in ePLM at 37° C. and 250 rpm and grown OD 1.0.
8. 20% Arabinose (0.01 volumes) was added to induce Phage production for 2 hours.
9. Lysed cultures were transferred to 15 mL Eppendorf conical tubes
10. Chloroform ( 1/40 volume) was added to the reaction to kill residual cells.
11. Phage lysate was spun down at 5000 rpm for 10 minutes at 4° C.
12. Phage lysate was transferred (avoid cell pellet) to a new 15 mL Eppendorf tube.
13. Phage lysate can then be used directly or stored at 4° C. for 1 year and indefinitely at −80° C.
14. ISA372 was grown overnight in LB with 1% glucose and Kan media.
15. ISA372 was then inoculated to 30 mL ePLM (1:100 dilution) and grown at 37° C. and 250 rpm to OD 1.0.
16. The cells were then harvested at 5000 rpm for 5 minutes and resuspended in 10 mL ePLM.
17. 1 mL Phage lysate was used to infect 1 mL of ISA372 from step 16.
18. Infection reaction was inoculated into 25 mL LB with Cm and Kan. Uninfected cells were killed with Cm and in this step.
19. Steps 4-6 were repeated once for a total of 2 AIDE mutagenesis cycles. No selection conducted between mutagenesis cycles.
20. Cultures were plated in LB+Cm agar plates to look for higher fluorescent colonies.
21. Steps 7-13 were repeated concurrently with step 20.
22. Phage lysates produced from cycle 2 was used to infect wild type E. coli C600.
23. Infection reactions were inoculated into 25 mL LB with Cm and grown overnight.
24. Saturated cultures were plated in LB+Cm agar plates to look for higher fluorescent colonies.
25. 21 colonies were picked and run in flow cytometry with negative (wild type cells) and positive (unevolved phagemid) controls.
26. sfGFP and pSC101 were then amplified from these colonies and sequenced by Sanger sequencing.
27. Detected mutations were reintroduced via NEB Q5@ Site-Directed Mutagenesis Kit to confirm phenotypes.
28. Cloned phagemids with the specific mutations were then run in flow cytometry with negative (wild type cells) and positive (unevolved phagemid) controls.

### Example—Heterologous Tagatose Consumption

Referring to FIGS. 6A and 6C, AIDE is applied to improve the ability of a heterologous tagatose consumption pathway to enable tagatose consumption in E. coli based on the above experiments demonstrate the overall concept and feasibility of AIDE to mutate and evolve large DNA segments in bacteria. A degradation pathway for tagatose (FDA approved sweetener) is evolved for probiotic applications. It has been shown that E. coli grows poorly in tagatose. It is intended to enable E. coli to consume tagatose efficiently by evolving a 5-gene tagatose catabolism pathway (from Bacillus licheniformis). Probiotic strains are developed that can make use of this carbon source and preferentially grow when tagatose is consumed. Tagatose could potentially therefore control the residence time of this probiotic strain. Tagatose consumption would lead to maintenance of the probiotic in the gut, whereas elimination of tagatose from the diet would lead to the reduction of probiotic abundance in the gut.

To demonstrate AIDE, a 5-gene tagatose catabolism pathway from Bacillus licheniformis in E. coli, resulting in clones with 65% shorter lag times during growth on tagatose after only two rounds of evolution. AIDE was applied to improve a heterologous tagatose consumption pathway in E. coli (FIG. 6C). E. coli C600 natively consumes tagatose poorly, taking over 12.5 hours to reach an optical density (OD) of 0.25 in media containing tagatose as a sole carbon source from an initial OD of 0.05. We chose a five-gene tagatose consumption pathway from Bacillus licheniformis for insertion in PM (FIG. 6D)(Van der Heiden, E. et al., Appl. Environ. Microbiol. 79:3511-3515 (2013)). This pathway includes orf48 (encoding a predicted transcriptional regulator in the murR rpiR family), fruA2 and orf51 (encoding a predicted phosphotransferase system that transports D-tagatose into the cell and converts it to tagatose 1-phosphate), fruK2 (encoding a predicted kinase that converts tagatose 1-phosphate to tagatose 1,6-bisphosphate), and gatY (encoding a predicted aldolase that converts tagatose 1,6-bisphosphate to dihydroxyacetone phosphate and D-glyceraldehyde 3-phosphate. This pathway is therefore a good model, as it encodes different functions that collectively elicit the phenotype of interest. Insertion of this pathway into PM yielded a C600 strain with a 29% reduction in lag time, taking 8.8 hours to achieve an OD of 0.25 in tagatose media. We therefore expected that evolving this pathway would lead E. coli to consume tagatose more efficiently. After 2 AIDE cycles comprising mutagenesis, growth-based selection, and transfer to fresh cells (FIG. 6C), we identified variants that grew faster in tagatose media than strains containing the unmutated pathway (FIG. 6D). All of these variants exhibited some combination of higher optical density (strain E3 exhibited a 2.6-fold higher cell density at 500 minutes than a strain containing the unmutated pathway) and reduced lag time (strain E3 exhibited a 64% reduction in time to reach an optical density of 0.25 than a strain containing the unmutated pathway).

### Step by Step Diversification of Tagatose Pathway and Selection of Improved Variants

1. Tagatose pathway from Bacillus licheniformis was assembled into phagemid backbone (ISA012) resulting in tagatose phagemid (ISA179).
2. ISA179 was miniprepped and transformed to ISA372 forming ISA374.
3. ISA374 was grown overnight in LB with 1% glucose and Kan and Cm at 37° C. and 250 rpm.
4. Cells were then harvested at 5000 rpm for 5 minutes and washed with 1×PBS.
5. Washed cells were inoculated into fresh LB media with Kan and Cm.
6. Mutagenesis was induced starting at OD 0.01 with 200 ng/mL aTc for 16 hours.
7. Evolved culture was sub-inoculated to OD 0.1 in ePLM at 37° C. and 250 rpm and grown OD 1.0.
8. 20% Arabinose (0.01 volumes) was added to induce Phage production for 2 hours.
9. Lysed cultures were transferred to 15 mL Eppendorf conical tubes
10. Chloroform ( 1/40 volume) was added to the reaction to kill residual cells.
11. Phage lysate was spun down at 5000 rpm for 10 minutes at 4° C.
12. Phage lysate was transferred (avoid cell pellet) to a new 15 mL Eppendorf tube.
13. Phage lysate can then be used directly or stored at 4° C. for 1 year and indefinitely at −80° C.
14. E. coli C600 strain containing P1kc:10 kb (ISA222) was grown overnight in LB media.
15. ISA222 was then inoculated to 30 mL ePLM (1:100 dilution) and grown at 37° C. and 250 rpm to OD 1.0.
16. The cells were then harvested at 5000 rpm for 5 minutes and resuspended in 10 mL ePLM.
17. 1 mL Phage lysate was used to infect 1 mL of ISA222 from step 15.
18. Infection reaction was inoculated into 25 mL LB+Cm. Uninfected cells were killed with Cm in this step.
19. Cells were washed with tagatose minimal media and then inoculated to 50 ml tagatose minimal media to starting OD 0.01.
20. Cells were at grown 37° C. and 250 rpm for 24 hours and then subinoculated into pre-warmed fresh tagatose media (3 times)
21. After 3^{rd} round selection, cell culture was sub-inoculated for phage production (Steps 7-13).
22. Phage lysate from first AIDE cycle was then used to infect ISA372.
23. Infection reaction was inoculated into 25 mL LB with Cm and Kan. Uninfected cells were killed with Cm and in this step.
24. Steps 3-21 were repeated once for a total of 2 AIDE cycles.
25. Phage lysates produced from each cycle before and after selection with controls (no mutagenesis induced) was used to infect wild type E. coli C600.
26. Infection reactions were then plated in tagatose minimal media agar plates.
27. Colonies that grew fast in the plates were inoculated and grown in the plate reader for growth curves analysis.
28. Colonies that had higher growth rates were stocks and grown in LB+Cm media for plasmids extraction.
29. Tagatose pathway from the isolated colonies were amplified via PCR and sequenced by sanger sequencing.
30. Pathways that show apparent growth benefits, were cloned into wildtype phagemid backbone and analyzed again.

TABLE 2
Mutations detected in Tagatose pathway after
AIDE cycles. RBS indicates mutations
were detected in the ribosome binding site

| Amino Acid | | | |
|---|---|---|---|
| Change | Sequence | Gene | Isolate |
| V56A | GTT --> GCT | murR/rpiR | E3 |
| Q127K | CAA --> AAA | murR/rpiR | E1 |
| RBS | A --> G | fruK2 | E3, E4 |
| P47P | CCA --> CCC | fruK2 | E2 |
| R149R | AGA --> AGG | fruK2 | E3 |
| A183T | GCA --> ACA | fruA2 | E1 |
| V428A | GTA --> GCA | fruA2 | E2 |
| A39A | GCC --> GCT | gatY | E2 |
| Y55D | TAT --> GAT | gatY | E2 |
| P255L | CCA --> CTA | gatY | E1 |

### Detailed Methods for the Above Examples

Strains and Media: E. coli C600 (CGSC 5394 C600).

Phage Infection: Overnight culture grown in LB with appropriate antibiotics was subioculated into ePLM (1:100 dilution). At OD 1, the cells were spun down at 5000 rpm for 5 minutes. The supernatant was discarded and the pellet was resuspended in 13 volume fresh ePLM. The cells were then added to phage lysate in a culture tube. The infection mixture was then incubated in a 37° C. shaking incubator for 20 minutes and then moved to a 37° C. standing incubator for 20 minutes. The infection mixture was quenched with an equal volume of Super Optimal Broth (SOC) containing 200 mM sodium citrate. The mixture was then incubated for 40 minutes in a 37° C. shaking incubator before being inoculated to 50 ml LB media with appropriate antibiotics or plated in LB agar plates containing appropriate antibiotics.

Phage Production: Overnight cultures of strains containing P1 and phagemid were subinoculated 100× in ePLM with appropriate antibiotics. At OD 0.8 cell cultures were induced with 20% L-arabinose ( 1/100 culture volume) and put back to the shaking incubator. After 2 hours the cultures were removed from the incubator and transferred to 15 ml centrifuge tubes containing chloroform. The tubes were left in ice for 5 minutes with gentle mixing or pipetting every minute. The tubes were then centrifuged at 5000 rpm for 10 minutes at 4 C. Produced phage was then transferred to sterile tubes for storage. Phage lysate is stable at 4 C for 1 year and indefinitely at −80 C.

Cloning: NEB Hifi 2× was used for Gibson assembly and NEB Q5 SDM was used to introduce point mutations.

Cell growth phase: 3 overnight E. coli C600 colonies grown in LB were inoculated to 50 ml PLM media and infected with spun down to obtain the same cell count for culture that were grown to OD 0.5, 1.0 and 1.5. The media was discarded and cells were resuspended with PLM and infected with lysate produced from ISA199 (E. coli C600 containing P1kc and 12 kbp phagemid)(See Table 3). Infected cultures were plated in LB/Cm plates for CFU/cell/ml count.

Media composition: Phage Lysate Media (PLM) is 100 mM MgCl₂ and 5 mM CaCl₂) added to lysogeny broth (LB). Enhanced PLM (ePLM) is 140 mM MgCl₂ and 7 mM CaCl₂) added to lysogeny broth (LB).

Engineered P1: P1Δcoi::KanR and P1:10 kb::KanR were a kind gift from Dr. Chase Beisel (North Carolina State University), and using FLP recombinase KanR was knocked out.

Flip recombinase experiment: Strains containing P1kcΔcoi::KanR and P1kc:10 kb::KanR were transformed with pCP20 and plated in LB+Ampicillin plates at 30° C. Grown colonies were inoculated into 5 mL of LB and grown overnight at 43° C. to induce FLP recombinase expression. Colonies were then screened for loss of KanR from phage genome.

Phagemid size infection rate: 8.0 kb, 12.8 kbp and 24.0 kbp and 42 kbp phagemids (Addgene #40784, #69669, and #40782) were transformed to ISA222. Phage lysate was produced from each strain and then used to infect wild type E. coli C600. Infected strains were plated in LB+Cm plates for CFU/ml counts. FIG. 5A shows the infection rate.

Single and double Stop codon reversion: CmR was inserted to a phagemid backbone with AmpR using Gibson assembly. Single and double mutations were introduced via Q5 Site-Directed Mutageneis to make stop codons in CmR. The phagemid with a dysfunctional CmR was cloned to a E. coli C600 strain containing MP6 and P1 phage (ISA308, ISA311, and ISA363). 3 biological replicates of the strains were grown overnight in LB containing 1% glucose and the appropriate antibiotics (Kan/Amp). The overnight cultures were plated on LB/Cm agar plates to check for escapers and background aTc-MP activity. The cultures were spun down and washed with 1×PBS to remove the residual glucose from the media. Washed cells were then inoculated to LB media containing Kan/Amp (1:1000) and with or without 200 ng/ml aTc to induce aTc-MP. The cultures were plated after 8 hours and 16 hours of induction in LB/Cm agar plates to check stop codon reversion. CFU/ml were counted the next day and random colonies were picked to check stop codon reversions.

pSC101-sfGFP Evolution: pSC101 and sfGFP were inserted in a phagemid backbone via Gibson assembly. pSC101-sfGFP phagemid was then transformed into E. coli C600 containing aTc-MP and P1::10 kb. The strain was evolved 2 times before selection to find the mutations that increased GFP production. Colonies that had brighter GFP were picked, sequenced and run on flow cytometry to quantify GFP production. Mutations that were found in pSC101 were introduced to unevolved phagemid via Q5 SDM to test the viability of the mutations and run again on flow cytometry.

Tagatose evolution: The tagatose pathway from Bacillus licheniformis (ATCC® 14580™) was inserted into the phagemid backbone via Gibson assembly. The phagemid was then transformed into E. coli C600 containing aTc-MP and P1kc:10 kb. The resulting strain was evolved in two rounds of diversification and selection as shown in FIG. 6C. The resulting phage lysate after two rounds of evolution was used to infect wild-type E. coli C600. The infected cells were plated on tagatose minimal media plates and colonies with bigger morphology were picked and grown overnight in LB media containing chloramphenicol (34 μg/mL). The cultures were then washed with tagatose minimal media (see methods: growth curves) and grown for 40 hours on a plate reader. The pathways in the colonies with faster growth rates were sequenced via sanger sequencing. The resulting mutations were then reintroduced to the unevolved phagemid via Q5 SDM and analyzed again (FIG. 6D).

Growth curves: Three (3) biological replicates of each isolate were grown overnight cultures in 96-deep-well plates (VWR International) were spun down at 4000 rpm for 10 minutes to discard the media and then washed with tagatose minimal media to remove the residual LB media and spun again at 4000 rpm for 10 minutes. The media was discarded, and the cells were resuspended with tagatose minimal media again. The cells were then subinoculated to 96-well-plates (Costar) (1:200 dilution) and grown for 40 hours in a plate reader (BioTek Synergy™ H1, Shake Mode: Double Orbital, Orbital Frequency: continuous shake 365 cpm, Interval: 10 minutes)

Infecting different E. coli Strains: P1kc:10 kb::KanR lysate produced from E. coli C600 (diversification strain) was used to infect wild type E. coli C600, MG1655, and Nissle 1917. Infection reactions were plated in LB+kan agar plates. P1 stability in these strains was checked via PCR. 12 kbp phagemid was transformed to the 3 strains containing P1kc:10 kb::KanR. 3 biological replicates from the transformed strain were grown overnight in LB+Cm and used for phage production. Phage lysate from each strain was used to infect wild type E. coli C600, MG1655, and Nissle 1917. FIG. 5D shows the infection rate for each route.

TABLE 3
Strains used in this study

| ISA number | Strain | plasmid description |
|---|---|---|
| ISA012 | DH10B | p15a, CamR, phagemid backone (addgene #40782) |
| ISA199 | E. coli C600 | P1kc and 12 kbp phagmeid p15a, CamR |
| ISA222 | E. coli C600 | E. coli C600 containing P1kc::10 kb |
| ISA265 | DH10B | pTet, tetR, DNAQ926, dam, seqA, emrR, ugi, cda1, CloDF13, KanR |
| (aTc-MP) | | |
| ISA308 | E. coli C600 | P1kc, aTc-MP, ampR phagemid with defective CmR (1 premature stop codon) |
| ISA311 | E. coli C600 | P1kc, aTc-MP, ampR phagemid with defective CmR (1 premature stop codon) |
| ISA363 | E. coli C600 | P1kc, aTc-MP, ampR phagemid with defective CmR (2 premature stop codon) |
| ISA372 | E. coli C600 | P1kc::10 kb, aTC-MP, KanR |
| ISA374 | E. coli C600 | P1kc::10 kb, aTC-MP, KanR, Tagatose phagemid, CmR, p15A |
| ISA384 | E. coli C600 | P1kc::10 kb, aTC-MP, KanR, sfGFP, p15A, CmR, phagemid backone |
| ISA410 | DH10B | sfGFP, pSC101, CmR, phagemid backone |
| ISA426 | E. coli C600 | P1kc::10 kb, aTC-MP, KanR, sfGFP, pSC101, CmR, phagemid backone |

TABLE 4
Sequences Showing Reversions of
Two Stop Codons Simultaneously
in a Chloramphenicol Resistance
Gene (see bold)

CmR double stop-SEQ ID NO: 1
Atggagaaaaaaatcactggatataccaccgttgatatat
cccaatgacatcgtaaagaacattttgaggcatttcagtc
agttgctcaatgtacctataaccagaccgttcagctggat
attacggcctttttaaagaccgtaaagaaaaataagcaca
agttttatccggcctttattcacattcttgcccgcctgat
gaatgctcatccggaatttcgtatggcaatgaaagacggt
gagctggtgatatgagatagtgttcacccttgttacaccg
ttttccatgagcaaactgaaacgttttcatcgctctggag
tgaataccacgacgatttccggcagtttctacacatatat
tcgcaagatgtggcgtgttacggtgaaaacctggcctatt
tccctaaagggtttattgagaatatgtttttcgtctcagc
caatccctgggtgagtttcaccagttttgatttaaacgtg
gccaatatggacaacttcttogcccccgttttcaccatgg
gcaaatattatacgcaaggcgacaaggtgctgatgccgct
ggcgattcaggttcatcatgccgtttgtgatggcttccat
gtcggcagaatgcttaatgaattacaacagtactgcgatg
agtggcagggcggggcgtaa
CmR reversion mutant 1-SEQ ID NO: 2
Atggagaaaaaaatcactggatataccaccgttgatatat
cccaaCgacatcgtaaagaacattttgaggcatttcagtc
agttgctcaatgtacctataaccagaccgttcagctggat
attacggcctttttaaagaccgtaaagaaaaataagcaca
agttttatccggcctttattcacattcttgcccgcctgat
gaatgctcatccggaatttcgtatggcaatgaaagacggt
gagctggtgatatgGgatagtgttcacccttgttacaccg
ttttccatgagcaaactgaaacgttttcatcgctctggag
tgaataccacgacgatttccggcagtttctacacatatat
tcgcaagatgtggcgtgttacggtgaaaacctggcctatt
tccctaaagggtttattgagaatatgtttttcgtctcagc
caatccctgggtgagtttcaccagttttgatttaaacgtg
gccaatatggacaacttcttcgcccccgttttcaccatgg
gcaaatattatacgcaaggcgacaaggtgctgatgccgct
ggcgattcaggttcatcatgccgtttgtgatggcttccat
gtcggcagaatgcttaatgaattacaacagtactgcgatg
agtggcagggcggggcgtaa
CmR reversion mutant 2-SEQ ID NO: 3
Atggagaaaaaaatcactggatataccaccgttgatatat
cccaaCgacatcgtaaagaacattttgaggcatttcagtc
agttgctcaatgtacctataaccagaccgttcagctggat
attacggcctttttaaagaccgtaaagaaaaataagcaca
agttttatccggcctttattcacattcttgcccgcctgat
gaatgctcatccggaatttcgtatggcaatgaaagacggt
gagctggtgatatgGgatagtgttcacccttgttacaccg
ttttccatgagcaaactgaaacgttttcatcgctctggag
tgaataccacgacgatttccggcagtttctacacatatat
tcgcaagatgtggcgtgttacggtgaaaacctggcctatt
tccctaaagggtttattgagaatatgtttttcgtctcagc
caatccctgggtgagtttcaccagttttgatttaaacgtg
gccaatatggacaacttcttcgcccccgttttcaccatgg
gcaaatattatacgcaaggcgacaaggtgctgatgccgct
ggcgattcaggttcatcatgccgtttgtgatggcttccat
gtcggcagaatgcttaatgaattacaacagtactgcgatg
agtggcagggcggggcgtaa
CmR reversion mutant 3-SEQ ID NO: 4
Atggagaaaaaaatcactggatataccaccgttgatatat
cccaaCgacatcgtaaagaacattttgaggcatttcagtc
agttgctcaatgtacctataaccagaccgttcagctggat
attacggcctttttaaagaccgtaaagaaaaataagcaca
agttttatccggcctttattcacattcttgcccgcctgat
gaatgctcatccggaatttcgtatggcaatgaaagacggt
gagctggtgatatgGgatagtgttcacccttgttacaccg
ttttccatgagcaaactgaaacgttttcatcgctctggag
tgaataccacgacgatttccggcagtttctacacatatat
tcgcaagatgtggcgtgttacggtgaaaacctggcctatt
tccctaaagggtttattgagaatatgtttttcgtctcagc
caatccctgggtgagtttcaccagttttgatttaaacgtg
gccaatatggacaacttcttcgcccccgttttcaccatgg
gcaaatattatacgcaaggcgacaaggtgctgatgccgct
ggcgattcaggttcatcatgccgtttgtgatggcttccat
gtcggcagaatgcttaatgaattacaacagtactgcgatg
agtggcagggcggggcgtaa

### REFERENCES

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.
1—Ellefson, J. W., Meyer, A. J., Hughes, R. A., Cannon, J. R., Brodbelt, J. S., & Ellington, A. D. (2014). Directed evolution of genetic parts and circuits by compartmentalized partnered replication. Nature Biotechnology, 32(1), 97.
2—Esvelt, K. M., Carlson, J. C., & Liu, D. R. (2011). A system for the continuous directed evolution of biomolecules. Nature, 472(7344), 499.
3—Halperin, S. O., Tou, C. J., Wong, E. B., Modavi, C., Schaffer, D. V., & Dueber, J E. (2018). CRISPR-guided DNA polymerases enable diversification of all nucleotides in a tunable window. Nature, 560(7717), 248.
4—Wrenbeck, E. E., Klesmith, J. R, Stapleton, J. A., Adeniran, A., Tyo, K. E., & Whitehead, T. A. (2016). Plasmid-based one-pot saturation mutagenesis. Nature methods, 13(11), 928.
5—Badran, A. H., & Liu, D. R. (2015). Development of potent in vivo mutagenesis plasmids with broad mutational spectra. Nature communications, 6, 8425.
6—Kittleson, J. T., DeLoache, W., Cheng, H. Y., & Anderson, J. C. (2012). Scalable plasmid transfer using engineered P1-based phagemids. ACS synthetic biology, 1(12), 583-589
7—Nafisi, P. M., Aksel, T., & Douglas, S. M. (2018). Construction of a novel phagemid to produce custom DNA origami scaffolds. Synthetic Biology, 3(1), ysy015.
Negative selection and stringency modulation in continuous evolution systems; U.S. Pat. No. 10,179,911.
VECTOR-BASED MUTAGENESIS SYSTEM; U.S. Patent Publication number: 20180087046.
Apparatus for continuous directed evolution of proteins and nucleic acids; Patent number: U.S. Pat. No. 9,771,574.
CONTINUOUS DIRECTED EVOLUTION; U.S. Patent Publication number: 20170009224.
NEGATIVE SELECTION AND STRINGENCY MODULATION IN CONTINUOUS EVOLUTION SYSTEMS; U.S. Patent Publication number: 20160348096.
Evolution of bond-forming enzymes; U.S. Pat. No. 9,267,127
CONTINUOUS DIRECTED EVOLUTION OF PROTEINS AND NUCLEIC ACIDS; U.S. Patent Publication number: 20150275202.
Continuous directed evolution of proteins and nucleic acids; U.S. Pat. No. 9,023,594.
EVOLUTION OF BOND-FORMING ENZYMES; U.S. Patent Publication number: 20140057317.
CONTINUOUS DIRECTED EVOLUTION; U.S. Patent Publication number: 20130345064.
EVOLVING NEW MOLECULAR FUNCTION; U.S. Patent Publication number: 20120165228.
Evolving new molecular function, U.S. Pat. No. 8,206,914.
Evolving new molecular function, U.S. Pat. No. 7,998,904.
Evolving New Molecular Function, U.S. Patent Publication number: 20110190141.
CONTINUOUS DIRECTED EVOLUTION OF PROTEINS AND NUCLEIC ACIDS, U.S. Patent Publication number: 20110177495.
Directed evolution of proteins, U.S. Pat. No. 7,807,408.
Evolving new molecular function, U.S. Pat. No. 7,771,935.
IN VIVO SELECTION SYSTEM FOR ENZYME ACTIVITY, U.S. Patent Publication number: 20090263851.
Evolving new molecular function, U.S. Pat. No. 7,557,068.
Evolving new molecular function, U.S. Pat. No. 7,491,494.
In vivo selection system for enzyme activity, U.S. Pat. No. 7,476,500.
Evolving new molecular function, U.S. Pat. No. 7,442,160.
Evolving new molecular function, U.S. Pat. No. 7,223,545.
Evolving new molecular function, U.S. Pat. No. 7,070,928.
Evolving new molecular function, U.S. Patent Publication number: 20040180412.
Evolving new molecular function, U.S. Patent Publication number: 20030113738.

It will be understood that various details of the presently disclosed subject matter can be changed without departing from the scope of the presently disclosed subject matter. Furthermore, the foregoing description is for the purpose of illustration only, and not for the purpose of limitation.

1. A method for directed evolution in a microbe, the method comprising:
introducing into a first host cell a propagation deficient phage genome and a vector comprising a target gene sequence to be mutated and a phage propagation component responsive to induce lysis of the first host cell and to provide for propagation of the phage genome;
exposing the first host cell to a mutagenesis agent;
inducing lysis of the first host cell and phage propagation to produce a lysate comprising phage particles comprising the target gene sequence; and
infecting a second host cell with the lysate.

2. The method of claim 1, wherein the first host cell and the second host cell are each a bacterial cell.

3. The method of claim 1, wherein the phage genome comprises a temperate phage genome.

4. The method of claim 3, wherein the temperate phage genome is a P1 phage genome.

5. The method of claim 1, wherein said first host cell further comprises a second vector comprising an inducible mutagenesis sequence.

6. The method of claim 1, wherein the mutagenesis agent is selected from the group consisting of nucleotide analogues, nucleoside precursors, alkylating agents, cross-linking agents, genotoxins, and radiation.

7. The method of claim 1, wherein the mutagenesis agent is a chemical mutagen.

8. The method of claim 1, further comprising screening for a selected function of a mutated target sequence.

9. The method of claim 8, wherein the step of screening comprises at least one of a bacteriophage display system, an antibiotic resistance and an expression of a reporter gene.

10. The method of claim 1, further comprising expressing an evolved protein or nucleic acid encoded by a mutated target sequence.

11. The method of claim 1, further comprising isolating a mutated target sequence.

12. The method of claim 1, wherein the method further comprises repeating said steps.

13. A system or kit configured for carrying out a method in accordance with claim 1.

14. A kit for directed evolution in a microbe, the kit comprising: a propagation deficient phage genome and a phage propagation component responsive to induce lysis of a first host cell and to provide for propagation of the phage genome.

15. The kit of claim 14, comprising one or more of the following: a vector for a target gene sequence to be mutated, a first host cell for the propagation deficient phage genome and the phage propagation component, a second host cell for a lysate comprising phage particles, and a mutagenesis agent.

16. The kit of claim 15, wherein the first host cell and the second host cell are each a bacterial cell.

17. The kit of claim 14, wherein the phage genome comprises a temperate phage genome.

18. The kit of claim 14, wherein the temperate phage genome is a P1 phage genome.

19. The kit of claim 15, comprising a second vector comprising an inducible mutagenesis sequence.

20. The kit of claim 15, wherein the mutagenesis agent is selected from the group consisting nucleotide analogues, nucleoside precursors, alkylating agents, cross-linking agents, and genotoxins.

21. The kit of claim 14, further comprising instructional material for a directed evolution method.
